(19) United States
(12) Patent Application Publication
(10) Pub. No.: US 2005/0049578 A1
(43) Pub. Date: Mar. 3, 2005

(54) Implantable ocular pump to reduce intraocular pressure

(76) Inventors: Hosheng Tu, Newport Coast, CA (US); David Haffner, Mission Viejo, CA (US); Morteza Gharib, San Marino, CA (US); Denis Phares, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/910,962

(22) Filed: Aug. 4, 2004

Related U.S. Application Data
(63) Continuation-in-part of application No. 10/636,797, filed on Aug. 7, 2003.
Application No. 10/636,797 is a continuation-in-part of application No. 10/395,472, filed on Mar. 21, 2003, now abandoned.
Application No. 10/395,472 is a continuation-in-part of application No. 09/549,350, filed on Apr. 14, 2000, now Pat. No. 6,638,239.

(51) Int. Cl. A61K 9/22

(52) U.S. Cl. 604/890.1

(57) ABSTRACT

A trabecular pump is implantable in the eye to reduce intraocular pressure. The pump drains aqueous humor from the anterior chamber into outflow pathways, such as Schlemm's canal. A feedback system includes the intraocular pump and a pressure sensor in communication with the pump, for regulating intraocular pressure.

### CROSS REFERENCE TO RELATED APPLICATION

This application is a continuation-in-part application of U.S. patent application Ser. No. 10/636,797 filed on Sep. 7, 2003, which is a continuation-in-part application of U.S. patent application Ser. No. 10/395,472 filed on Mar. 21, 2003, now abandoned, which is a continuation-in-part application of U.S. patent application Ser. No. 09/549,350 filed on Apr. 14, 2000, now U.S. Pat. No. 6,638,239. Each of these patent applications and patents is incorporated herein in its entirety by reference.

### FIELD OF THE INVENTION

The invention generally relates to medical devices and methods for the reduction of elevated pressure in organs of the human body. More particularly, the invention relates to the treatment of glaucoma by reducing intraocular pressure to a desired level.

### BACKGROUND OF THE INVENTION

About two percent of people in the United States have glaucoma. Glaucoma is a group of eye diseases that causes pathological changes in the optic disk and corresponding visual field loss, resulting in blindness if untreated. Intraocular pressure elevation is a major etiologic factor in glaucoma.

In glaucomas associated with an elevation in eye pressure, the source of resistance to outflow is in the trabecular meshwork. The tissue of the trabecular meshwork normally allows aqueous humor (“aqueous”) to enter Schlemm's canal, which then empties into aqueous collector channels in the posterior wall of Schlemm's canal and then into aqueous veins. The aqueous is a transparent liquid that fills the region between the cornea at the front of the eye and the lens. The aqueous is constantly secreted by the ciliary body around the lens, so there is a continuous flow of aqueous humor from the ciliary body to the eye's anterior (front) chamber. The eye's pressure is determined by a balance between the production of aqueous and its exit through the trabecular meshwork (major route) or via uveal scleral outflow (minor route). The trabecular meshwork is located between the outer rim of the iris and the internal periphery of the cornea. The portion of the trabecular meshwork adjacent to Schlemm's canal causes most of the resistance to aqueous outflow (juxtacanilicular meshwork).

Glaucoma is principally classified into two categories: closed-angle glaucoma and open-angle glaucoma. Closed-angle glaucoma is caused by closure of the anterior angle by contact between the iris and the inner surface of the trabecular meshwork. Closure of this anatomical angle prevents normal drainage of aqueous humor from the anterior chamber of the eye. Open-angle glaucoma is any glaucoma in which the angle of the anterior chamber remains open, but the exit of aqueous through the trabecular meshwork is diminished. The exact cause for diminished filtration is unknown for most cases of open-angle glaucoma. However, there are secondary open-angle glaucomas, which can involve edema or swelling of the trabecular spaces (from steroid use), abnormal pigment dispersion, or diseases such as hyperthyroidism that produce vascular congestion.

Current therapies for glaucoma are directed at decreasing intraocular pressure. Initially, the intraocular pressure can be decreased by medical therapy with drops or pills that reduce the production of aqueous humor or increase the outflow of aqueous. However, these various drug therapies for glaucoma are sometimes associated with significant side effects, such as headache, blurred vision, allergic reactions, death from cardiopulmonary complications and potential interactions with other drugs. When drug therapy fails, surgical therapy is used. Surgical therapy for open-angle glaucoma comprise laser (trabeculoplasty), trabeculectomy, and aqueous shunting implants (after failure of trabeculectomy or if trabeculectomy is unlikely to succeed). Trabeculectomy is a major surgery that is widely used and is augmented with topically applied anticancer drugs such as 5-flurouracil or mitomycin-C to decrease scarring and increase surgical success.

Approximately 100,000 trabeculectomies are performed on Medicare-age patients per year in the United States. This number would increase if the morbidity associated with trabeculectomy could be decreased. The current morbidity associated with trabeculectomy consists of failure (10-15%), infection (a life long risk about 2-5%), choroidal hemorrhage (1%, a severe internal hemorrhage from excessively low pressure resulting in visual loss), cataract formation, and hypotony maculopathy (potentially reversible visual loss from excessively low pressure).

If it were possible to bypass the local resistance to outflow of aqueous at the point of the resistance and to use existing outflow mechanisms, surgical morbidity would greatly decrease due to the backpressure of the episcleral aqueous veins which would prevent the eye pressure from going too low. Avoiding excessive reductions of the eye pressure would virtually eliminate the risk of hypotony maculopathy and choroidal hemorrhage. Furthermore, visual recovery would be very rapid and risk of infection would be very small (a reduction from 2-5% to 0.05%). Because of these reasons, surgeons have tried for decades to develop a workable surgery for the trabecular meshwork.

The previous techniques that have been tried include goniotomy and trabeculotomy, and other mechanical disruptions of the trabecular meshwork, such as trabeculopuncture, goniophotoablation, laser trabecular ablation and goniocurretage. These techniques are briefly described below.

Goniotomy/Trabeculotomy: Goniotomy and trabeculotomy are simple and directed techniques of microsurgical dissection with mechanical disruption of the trabecular meshwork. These techniques initially had early favorable responses in the treatment of open-angle glaucoma. However, long-term review of surgical results showed only limited success in adults. In retrospect, these procedures probably failed secondary to repair mechanisms and a process of “filling-in.” The filling-in process is the result of a healing process that has the detrimental effect of collapsing and/or closing the created opening throughout the trabecular meshwork. Once the created openings close, the pressure builds back up and the surgery fails.

Trabeculopuncture: Q-switched Neodymium (Nd):YAG lasers also have been investigated for an optically invasive technique for creating full-thickness holes in the trabecular meshwork. However, the relatively small holes created by this trabeculopuncture technique also fail due to a filling-in effect.

Goniophotoablation/Laser Trabecular Ablation: Goniophotoablation is disclosed by Berlin in U.S. Pat. No. 4,846,172, incorporated herein in its entirety by reference, which describes the use of an excimer laser to treat glaucoma by ablating the trabecular meshwork. This technique was not demonstrated by clinical trial to succeed. Hill et al. used an Erbium:YAG laser to create full thickness holes through trabecular meshwork (Hill et al., Lasers in Surgery and Medicine 11:341-346, 1991), incorporated herein in its entirety by reference. This technique was investigated in a primate model and a limited human clinical trial at the University of California, Irvine. Although morbidity was zero in both trials, success rates did not warrant further human trials. Failure again was from filling-in of created defects in the trabecular meshwork by repair mechanisms. Neither of these techniques is an optimal surgical technique for the treatment of glaucoma.

Goniocurretage: This technique is an ab-interno (from the inside) mechanical disruptive technique. This technique uses an instrument similar to a cyclodialysis spatula with a microcurrette at the tip. Initial results are similar to trabeculotomy that fails secondary to repair mechanisms and a process of filling-in.

Although trabeculectomy is the most commonly performed filtering surgery, viscocanulostomy (VC) and non-penetrating trabeculectomy (NPT) are two new variations of filtering surgery. These techniques are ab-externo (from the outside), major ocular procedures in which Schlemm's canal is surgically exposed by making a large and deep scleral flap. In the VC procedure, Schlemm's canal is cannulated and viscoelastic substance injected (which dilates Schlemm's canal and the aqueous collector channels). In the NPT procedure, the inner wall of Schlemm's canal is stripped off after surgically exposing the canal.

Trabeculectomy, VC, and NPT are performed under a conjunctival and scleral flap, such that the aqueous humor is drained onto the surface of the eye or into the tissues located within the lateral wall of the eye. Normal physiological outflows are not used. These surgical operations are major procedures with significant ocular morbidity. When trabeculectomy, VC, and NPT are thought to have a low chance for success, a number of implantable drainage devices have been used to ensure that the desired filtration and outflow of aqueous humor through the surgical opening will continue. The risk of placing a glaucoma drainage implant also includes hemorrhage, infection and postoperative double vision that is a complication unique to drainage implants.

Examples of implantable shunts or devices for maintaining an opening for the release of aqueous humor from the anterior chamber of the eye to the sclera or space underneath conjunctiva have been disclosed in U.S. Pat. No. 6,007,511 (Prywes), U.S. Pat. No. 6,007,510 (Nigam), and U.S. Pat. No. 5,397,300 (Baerveldt et al.), each of which is incorporated herein in its entirety by reference.

The above embodiments and variations thereof have numerous disadvantages and moderate success rates. They involve substantial trauma to the eye and require great surgical skill by creating a hole over the full thickness of the sclera/cornea into the subconjunctival space. Furthermore, normal physiological outflow pathways are not used. The procedures are mostly performed in an operating room generating a facility fee, anesthesiologist's professional fee and have a prolonged recovery time for vision. The complications of filtration surgery have inspired ophthalmic surgeons to look at other approaches to lowering intraocular pressure.

### SUMMARY OF THE INVENTION

Some embodiments provide an apparatus for transporting aqueous humor from the anterior chamber of an eye. The apparatus comprises an inlet that receives aqueous humor from the anterior chamber. The apparatus further comprises an outlet that outputs aqueous humor to a location outside the anterior chamber. The apparatus further comprises a pump that pumps aqueous humor from the inlet to the outlet. The pump comprises a pair of substantially one-way valves that are spaced to provide a fluid chamber therebetween.

In some embodiments, the volume of the fluid chamber changes in response to a variation in intraocular pressure to drive the pump. In some embodiments, the pump is located between the inlet and the outlet. In some embodiments, the location outside the anterior chamber is within Schlemm's canal.

Some embodiments further include means for powering the pump, such as a power source coupled to the pump. Exemplary power sources include, but are not limited to, mechanical or electrical power sources. The pump of certain embodiments is driven by changes in intraocular pressure that result from at least one of blinking and arterial pulse, both of which cause variations in intraocular pressure.

Some embodiments comprise a method of pumping aqueous humor from the anterior chamber of an eye to a location outside the anterior chamber. The method comprises providing a fluid chamber having an inlet that receives aqueous humor from the anterior chamber. The method further comprises changing the volume of the fluid chamber such that the aqueous humor is pumped from the inlet to an outlet located outside the anterior chamber.

Some embodiments comprise an apparatus for transporting aqueous humor from the anterior chamber of an eye. The apparatus comprises an inlet that receives aqueous humor from the anterior chamber and an outlet that outputs aqueous humor to a location outside the anterior chamber. The apparatus further comprises a pump that pumps aqueous humor from the inlet to the outlet. The apparatus further comprises a sensor that senses intraocular pressure and provides a signal indicative of the sensed intraocular pressure. The pump is responsive to the signal to regulate flow through the pump.

In some embodiments, the sensor is electrically coupled to the pump. In some embodiments, the sensor is wirelessly coupled to the pump.

Certain embodiments include a method of regulating intraocular pressure. The method comprises implanting a micropump in the eye such that the pump pumps fluid from the anterior chamber to a location outside the anterior chamber. The method further comprises sensing intraocular pressure. The method further comprises using the sensed intraocular pressure to adjust a flow of the fluid through the pump. The sensing can be performed by a sensor in communication with the micropump.

In some embodiments, a trabecular pump stent has an inlet portion configured to extend through a portion of the trabecular meshwork of an eye. The pump stent further comprises an outlet portion configured to extend into Schlemm's canal of the eye. The inlet portion is disposed to the anterior chamber for aqueous communication between the anterior chamber and Schlemm's canal.

In some embodiments, the trabecular pump stent comprises an inlet portion which is configured to extend through a portion of the trabecular meshwork. The pump stent further comprises an outlet portion configured to extend into Schlemm's canal. The pump stent further comprises an anchoring means for stabilizing the pump stent in place. The anchoring means of certain embodiments comprises at least one protrusion which is configured to anchor through the trabecular meshwork into Schlemm's canal.

Some embodiments comprise an inlet portion configured to extend through a portion of the trabecular meshwork, an outlet portion configured to extend into Schlemm's canal, and means for controlling aqueous flow in one direction. The means for controlling aqueous flow and intraocular pressure may comprise an active method, such as a pump.

Certain embodiments provide a method for pumping fluid through a trabecular pump stent in one direction. The method comprises activating a pumping element that is mounted on the stent. The pumping element is powered by piezoelectricity converted from mechanical stress resulting from a physiological force selected from a group consisting of blink pressure pulses, ocular pressure pulses, body motions, head motions, and eye motions.

Certain embodiments provide a method for pumping fluid through a trabecular pump stent in one direction. The method comprises activating a pumping element that is mounted on the stent. The pumping element is powered by electricity converted from light power via a microphotodiode mechanism.

Certain embodiments provide a method for pumping fluid through a trabecular pump stent in one direction. The method comprises activating a pumping element that is mounted on the stent. The pumping element is powered by electricity converted from a temperature differential based on a thermo-electrical mechanism.

Certain embodiments provide a method for pumping fluid through a trabecular pump stent in one direction. The method comprises activating a pumping element that is mounted on the stent. The pumping element is powered by electricity converted from isotope energy via an isotope decay mechanism.

Certain embodiments provide a method for pumping fluid through a trabecular pump stent in one direction. The method comprises setting a target intraocular pressure level. The method further comprises sensing real-time intraocular pressure. The method further comprises comparing the sensed pressure to the target level. The method further comprises starting pumping aqueous out of an anterior chamber when the sensed pressure is higher than the target level.

Certain embodiments provide a trabecular pump stent for pumping fluid from an anterior chamber to Schlemm's canal. The pump stent comprises an inlet portion with an inlet terminal exposed to an anterior chamber. The pump stent further comprises an outlet portion with an outlet terminal exposed to Schlemm's canal. The pump stent further comprise a middle portion having a proximal end and a distal end. The pump stent further comprises a first check valve located at a proximal end of the middle portion and a second check valve located at a distal end of the middle portion.

Certain embodiments provide a pump for transporting aqueous fluid from the anterior chamber of an eye. The pump comprises an inlet configured to receive the aqueous fluid from the anterior chamber. The pump further comprises an adjustable volume fluidly coupled to the inlet. The pump further comprises an outlet fluidly coupled to the adjustable volume. The outlet is configured to output the aqueous fluid to a location outside the anterior chamber.

In certain further embodiments, the adjustable volume comprises a first tubular portion fluidly coupled to the inlet. The first tubular portion has a first fluidic characteristic. The adjustable volume further comprises a second tubular portion fluidly coupled to the first tubular portion. The second tubular portion has a second fluidic characteristic different from the first fluidic characteristic. The pump further comprises an actuator outside the adjustable volume. The actuator is configured to change the adjustable volume and to generate a pressure difference between the first tubular portion and the second tubular portion, wherein the pressure difference pumps the aqueous fluid from the inlet, through the adjustable volume, and through the outlet.

Certain embodiments provide a method of pumping aqueous fluid from the anterior chamber of an eye to a location outside the anterior chamber. The method comprises receiving aqueous fluid from the anterior chamber in a first portion of a pump. The method further comprising outputting the aqueous fluid from a second portion of the pump to the location outside the anterior chamber. The method further comprising adjusting a pump volume fluidly coupled to the first portion and fluidly coupled to the second portion. Adjusting the pump volume generates a pressure difference between the first portion and the second portion which pumps the aqueous fluid from the first portion to the second portion.

Certain embodiments provide a pump for transporting aqueous fluid from the anterior chamber of an eye. The pump comprises an inlet configured to receive the aqueous fluid from the anterior chamber. The pump further comprises an outlet configured to output the aqueous fluid to a location outside the anterior chamber. The pump further comprises a tubular conduit fluidly coupled to the inlet and fluidly coupled to the outlet. The pump further comprises at least one electrode configured to generate an electric field along and generally parallel to at least a portion of the tubular conduit. The electric field induces flow of aqueous fluid from the inlet to the outlet.

Certain embodiments provide a method of pumping aqueous fluid from the anterior chamber of an eye to a location outside the anterior chamber. The method comprises receiving aqueous fluid from the anterior chamber in a first portion of a pump. The method further comprises outputting the aqueous fluid from a second portion of the pump to the location outside the anterior chamber. The method further comprises generating an electric field generally along a direction from the first portion to the second portion. The electric field inducing a flow of the aqueous fluid from the first portion to the second portion.

### BRIEF DESCRIPTION OF THE DRAWINGS

Additional objects and features of certain embodiments of the present invention will become apparent from the following Detailed Description of Exemplary Embodiments, when read with reference to the accompanying drawings.

FIG. 1 schematically illustrates a sectional view of an eye.

FIG. 2 schematically illustrates a close-up sectional view, showing the anatomical diagram of trabecular meshwork and the anterior chamber of the eye.

FIGS. 3A, 3B, and 3C schematically illustrate the operation of a pressure-pulse-driven pump as an implanted trabecular stent.

FIG. 4 schematically illustrates one embodiment of the pressure-pulse-driven pump at a Schlemm's canal implant location.

FIG. 5 schematically illustrates another embodiment of the pressure-pulse-driven pump at an anterior-angle implant location.

FIG. 6 schematically illustrates an overpressure prevention mechanism.

FIG. 7 schematically illustrates an under-pressure protection mechanism.

FIG. 8 is a schematic block diagram illustrating a pump and sensor functions for controlling the intraocular pressure of an eye.

FIG. 9 schematically illustrates one embodiment of a pressure pulse-driven-pump implant.

FIGS. 10A, 10B, and 10C schematically illustrate the generation of electricity from mechanical stress using the piezoelectric effect of a PZT element.

FIGS. 11A, 11B, and 11C schematically illustrate the generation of mechanical motion from electrical signals using the piezoelectric effect of a PZT element.

FIG. 12 schematically illustrates the piezoelectric effect of a bimorph consisting of two thin PZT strips bonded together.

FIG. 13 schematically illustrates the placement of a PZT element in an eye for providing piezoelectricity to a trabecular pump.

FIG. 14 schematically illustrates the placement of a micro-photovoltaic cell arrangement in an eye for providing light energy to a trabecular pump.

FIGS. 15A-15D schematically illustrate various embodiments of a hydroelastic pump in an eye for controlling the target IOP.

FIG. 16 schematically illustrate a combined pump and valved stent system for maintaining desired IOP of a patient during the day and the night.

FIG. 17 schematically illustrates one embodiment of an electrokinetic pump implant for drainage of aqueous humor.

FIG. 18A schematically illustrates one embodiment of an electrokinetic pump element with a detached electrode placement and external power source.

FIG. 18B schematically illustrates one embodiment of an electrokinetic pump element with attached straight or annular electrode placement and external power source.

FIG. 18C schematically illustrates one embodiment of an electrokinetic pump element with attached flat electrode placement and attached power source.

FIG. 18D schematically illustrates one embodiment of an electrokinetic pump element with attached annular electrode placement and attached power source.

FIG. 19 schematically illustrates the placement of an electrokinetic pump in an eye for controlling the target IOP.

### DETAILED DESCRIPTION OF EXEMPLARY EMBODIMENTS

The trabecular meshwork and juxtacanilicular tissue together provide the majority of resistance to the outflow of aqueous and, as such, are logical targets for surgical removal in the treatment of open-angle glaucoma. In addition, minimal amounts of tissue are altered and existing physiologic outflow pathways are utilized. Trabecular bypass surgery has the potential for much lower risks of choroidal hemorrhage, infection and uses existing physiologic outflow mechanisms. This surgery could be performed under topical anesthesia in a physician's office with rapid visual recovery.

Therefore, there is a great clinical need for the treatment of glaucoma by a method that is faster, safer, and less expensive than currently available modalities. Trabecular bypass surgery is an innovative surgery that uses a micro stent, shunt, or other implant to bypass diseased trabecular meshwork alone at the level of trabecular meshwork and use or restore existing outflow pathways. Certain embodiments provide a means and methods for treating and controlling elevated intraocular pressure in a manner which is simple, effective, and disease site-specific with an implanted micropump and, in some cases, a remote or attached intraocular pressure (IOP) sensor.

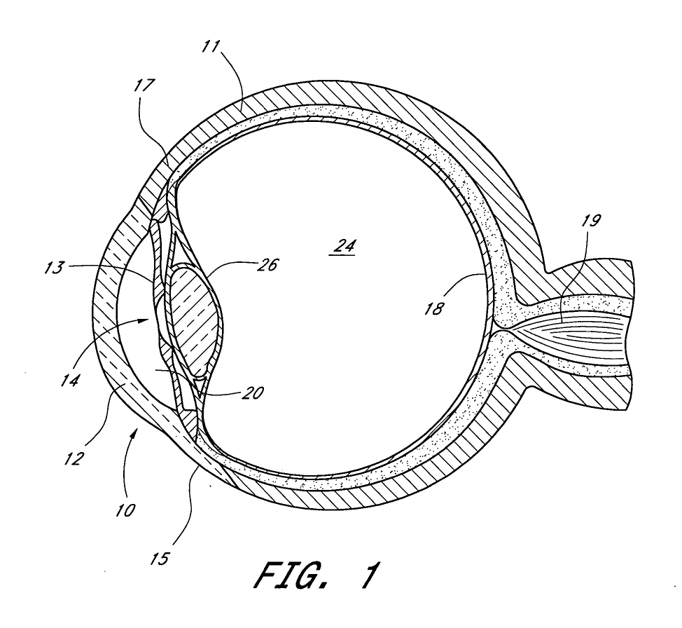

Referring to FIGS. 1 to 16, a trabecular pump is illustrated, which may be attached to or couple with a trabecular stent. In particular, a trabecular stent implant is used to bypass diseased trabecular meshwork. In certain embodiments, the trabecular stent implant has a pump and, in some embodiments, the implant also has a pressure sensor for controlling the intraocular pressure at a desired level.

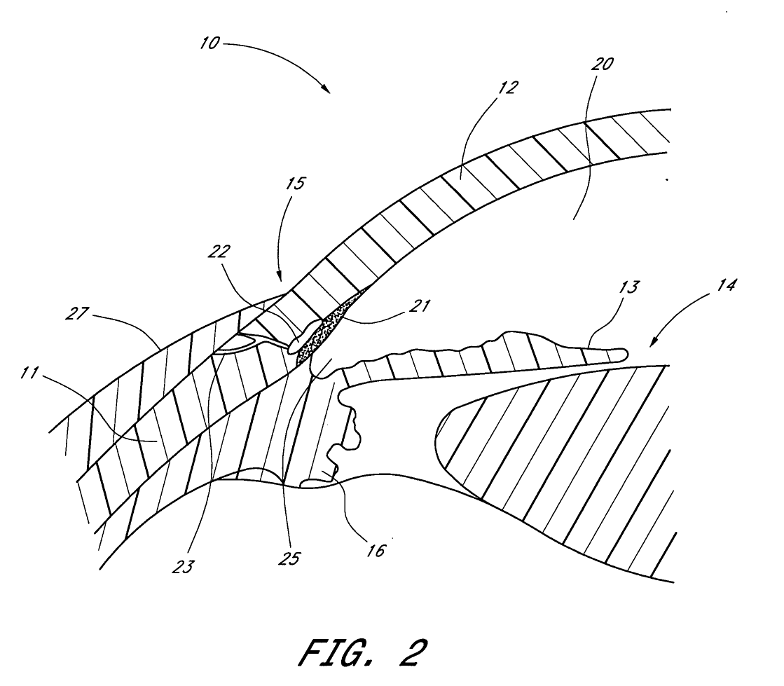

For background illustration purposes, FIG. 1 shows a sectional view of an eye 10, while FIG. 2 shows a close-up view, showing the relative anatomical locations of the trabecular meshwork 21, the anterior chamber 20, and Schlemm's canal 22. Thick collagenous tissue known as sclera 11 covers the entire eye 10 except that portion covered by the cornea 12. The cornea 12 is a thin transparent tissue that focuses and transmits light into the eye 10 and the pupil 14, which is the circular hole in the center of the iris 13 (colored portion of the eye 10). The cornea 12 merges into the sclera 11 at a juncture referred to as the limbus 15. The ciliary body 16 begins internally in the eye 10 and extends along the interior of the sclera 11 and becomes the choroid 17. The choroid 17 is a vascular layer of the eye underlying the retina 18. The optic nerve 19 transmits visual information to the brain and is progressively destroyed by glaucoma.

The anterior chamber 20 of the eye 10, which is bound anteriorly by the cornea 12 and posteriorly by the iris 13 and lens 26, is filled with aqueous humor or aqueous fluid 24. Aqueous fluid 24 is produced primarily by the ciliary body 16 and reaches the anterior chamber angle 25 formed between the iris 13 and the cornea 12 through the pupil 14. In a normal eye 10, the aqueous fluid 24 is removed through the trabecular meshwork 21. Aqueous fluid 24 passes through the trabecular meshwork 21 into Schlemm's canal 22 and through the aqueous veins 23, which merge with blood-carrying veins, and into venous circulation. Intraocular pressure of the eye 10 is maintained by the intricate balance of secretion and outflow of the aqueous fluid 24 in the manner described above. Glaucoma is characterized by the excessive buildup of aqueous fluid 24 in the anterior chamber 20, which produces an increase in intraocular pressure. Since fluids are relatively incompressible, the pressure is directed equally to all areas of the eye 10.

As shown in FIG. 2, the trabecular meshwork 21 constitutes a small portion of the sclera 11. It is understandable that creating a hole or opening for implanting a device through the tissues of the conjunctiva 27 and the sclera 11 is relatively a major surgery as compared to surgery for implanting a device through the trabecular meshwork 21 only.

In certain embodiments, a method increases aqueous humor outflow in an eye 10 of a patient to reduce the intraocular pressure therein. The method comprises bypassing diseased trabecular meshwork 21 at the level of the trabecular meshwork 21 and thereby restoring existing outflow pathways. In certain embodiments, a method for increasing aqueous humor outflow in an eye 10 of a patient to reduce an intraocular pressure therein comprises bypassing diseased trabecular meshwork 21 at a level of the trabecular meshwork 21 with a trabecular stent implant and using existing outflow pathways. The trabecular stent implant may be an elongated trabecular stent or other appropriate shape, size or configuration, with a micropump and/or a pressure sensor. In certain embodiments of an elongated trabecular stent implant, the trabecular stent has an inlet end, an outlet end, and a lumen therebetween, wherein the inlet end is positioned at an anterior chamber 20 of the eye 10 and the outlet end is positioned at about an exterior surface of the diseased trabecular meshwork 21. Furthermore, the outlet end may be positioned into fluid collection channels of the existing outflow pathways. Optionally, the existing outflow pathways may comprise Schlemm's canal 22. The outlet end may be further positioned into fluid collection channels up to the level of the aqueous veins 23, with the trabecular stent inserted either in a retrograde or antegrade fashion with respect to the existing outflow pathways.

In a further embodiment, a method for increasing aqueous humor outflow in an eye 10 of a patient to reduce an intraocular pressure therein comprises (a) creating an opening in the trabecular meshwork 21, wherein the trabecular meshwork 21 comprises an interior side and an exterior side; (b) inserting a trabecular pump stent into the opening; (c) activating a micropump on or in the trabecular pump stent; and (d) transporting the aqueous humor 24 by the trabecular pump stent to bypass the trabecular meshwork 21 at the level of the trabecular meshwork 21 from the interior side to the exterior side of the trabecular meshwork 21.

The trabecular stent implant may comprise a biocompatible material, such as a medical grade silicone, for example, the material sold under the trademark Silastic™, which is available from Dow Corning Corporation of Midland, Mich., or polyurethane, which is sold under the trademark Pellethane™, which is also available from Dow Corning Corporation. In an alternate embodiment, other biocompatible materials (biomaterials) may be used, such as polyvinyl alcohol, polyvinyl pyrolidone, collagen, heparinized collagen, tetrafluoroethylene, fluorinated polymer, fluorinated elastomer, flexible fused silica, polyolefin, polyester, polysilicon, mixture of biocompatible materials, and the like. In a further alternate embodiment, a composite biocompatible material by surface coating the above-mentioned biomaterial may be used, wherein the coating material may be selected from the group consisting of polytetrafluoroethylene (PTFE), polyimide, hydrogel, heparin, therapeutic drugs, and the like.

Control of intraocular pressure is the primary treatment modality for patients with ocular hypertension or glaucoma. Certain embodiments use a pump stent to achieve pressure control at a desired pressure level that is possibly lower than its downstream pressure. The pump stent may comprise a micropump or the like, preferably a valveless or bladeless pump in some embodiments. Alternatively, the pump may comprise a rotary, helical, or propeller blade pump design (not shown).

The pump of certain embodiments utilizes an energy source to move fluid from the anterior chamber 20 to Schlemm's canal 22 or other physiological outflow areas, for example, collector channels, aqueous veins 23, episcleral veins, sub-conjunctival spaces and any tissue area adjacent or surrounding the anterior chamber 20. Many sources of energy are available to drive the pump. By way of example, the energy sources may consist of ocular pressure pulse, blink pressure pulse, light power, body motion, head motion and eye motion, temperature differential (such as a thermoelectric generator), or stored energy (such as batteries). The pump of certain embodiments is implanted as a trabecular pump stent or mounted on or around a trabecular stent and utilizes the energy source that is disclosed. A “trabecular pump stent” is herein intended to mean a pump placed within the trabecular meshwork 21 that pumps fluid (for example, aqueous humor 24) from an anterior chamber 20 to Schlemm's canal 22 or downstream therefrom.

Implantable Pumps

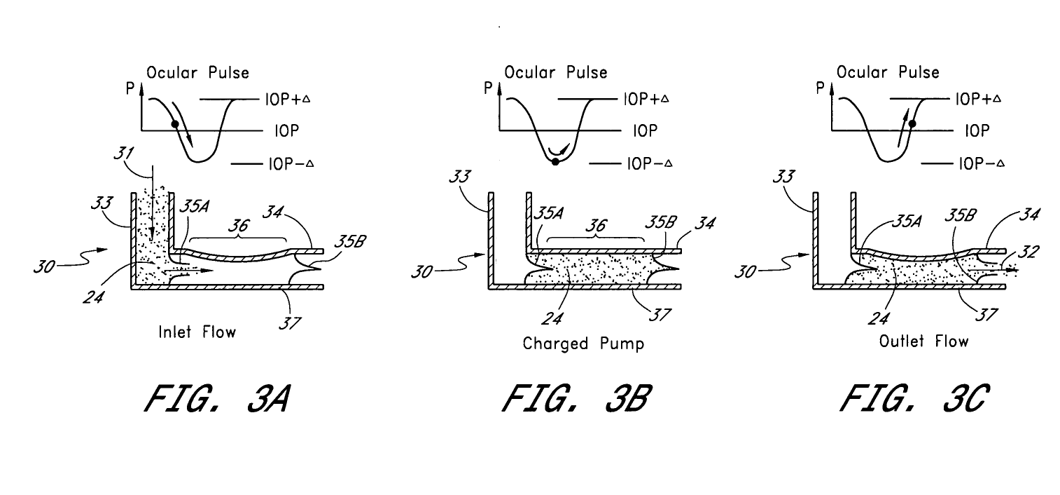

FIGS. 3A-3C schematically illustrate the operation of a simple pump 30 having an inlet portion 33 and an outlet portion 34. The pump 30 also has a compressible middle portion 36 and having two check valves 35A, 35B that are driven by pressure fluctuations in the eye 10. In certain embodiments, the middle portion 36 comprises a compressible tube 37. The energy source for causing the middle portion 36 to compress may comprise pressure fluctuations, such as ocular pressure pulse, blink pressure pulse and the like. The energy may be used directly or stored in a battery-type reservoir for future use to drive a compressing unit mounted on the middle portion 36. The pump entrance or inlet portion 33 is positioned in or connected to the anterior chamber 20. The pump exit or outlet portion 34 is positioned in or connected to Schlemm's canal 22 or any point downstream. In one embodiment, the inlet portion 33 and the outlet portion 34 are made of non-expandable, non-compressible material while the volume of the middle portion 36 can increase and decrease as a result of compression or expansion of the compressible tube 37.

U.S. Pat. No. 6,682,500 issued on Jan. 27, 2004, the entire contents of which are incorporated herein by reference, discloses an implantable, pressure adjustable diaphragm pump system which is compatible with embodiments described herein and which is characterized by a common type of actuating mechanism. A flexing ionic polymer conductor composite synthetic muscle, which is a type of ionic polymer metal composite synthetic muscle, functions as the primary actuator. The pump system may be effectively self-powered using a synthetic muscle attached to a local bending or twisting force.

In one embodiment, the ocular pressure pulse is used as an example in FIGS. 3A-3C. The upper parts of FIGS. 3A-3C show a single cycle in the repetitive ocular pulsations of the intraocular pressure. These cycles are often seen in tomographic pressure tracings with peak-to-peak amplitudes of about 1 to 3 mmHg. The ocular pulse is driven by the heart rate as the blood pressure varies from systole to diastole with each beat of the heart pumping. In the pressure tracings, the mean value is labeled as the IOP (intraocular pressure) of the eye 10 and pressure variations to peak and to valley are indicated by the symbol Δ. The black circles on the waveforms of FIGS. 3A-3C represent the cycle points in the operation of the pump 30.

There are three steps in this pumping process as schematically illustrated in FIGS. 3A-3C. In general, the pump 30 comprises an incompressible inlet portion 33, a compressible middle portion 36 located between a first check valve 35A and a second check valve 35B, and an incompressible outlet portion 34. In one embodiment, the inlet portion 33 may be compressible so long as the differential pressure between the inlet portion 33 and the middle portion 36 enables pushing aqueous fluid 24 through the first check valve 35A. In another embodiment, the outlet portion 34 may be compressible so long as the differential pressure between the middle portion 36 and the outlet portion 34 enables pushing aqueous fluid 24 through the second check valve 35B.

In the first step schematically illustrated in FIG. 3A, the inlet portion 33 of the pump body is filled with aqueous fluid 24 (as shown by an arrow 31). When the pressure rises and exceeds the opening pressure of the first check valve 35A, aqueous fluid 24 starts to flow into the middle portion 36 until the pressure equalizes between the inlet portion 33 and the middle portion 36. In the second step schematically illustrated in FIG. 3B, the tube 37 of the middle portion 36 is compressed. The compression of the tube 37 of the middle portion 36 can be achieved by any conventional means for pinching, wrapping around, or sandwiching with force. In certain embodiments, the tube 37 of the middle portion 36 is compressed by a mechanical pumping element using one of the energy sources. When the pressure in the middle portion 36 rises and exceeds the opening pressure of the second check valve 35B, the aqueous fluid 24 is pushed out through the outlet portion 34, as shown by an arrow 32 in FIG. 3C. Further, when the tube 37 of the middle portion 36 has expanded or reversed to its original size, the tube 37 is decompressed and sucks aqueous fluid 24 from the inlet portion 33 into the middle portion 36. This pump cycle repeats as the ocular pulse cycle continues. In this way, the pump 30 moves aqueous fluid 24 from the anterior chamber 20 to points downstream in the aqueous outflow system and the check valves 35A, 35B prevent reverse flow into the eye 10. The operating principles of a blink-pressure driven pump is similar, but is actuated at a larger pressure differential since blink-induced pressure changes are much larger than ocular pulse pressure variations.

Certain embodiments provide a pump 30 for pumping fluid from the anterior chamber 20 to Schlemm's canal 22 or downstream therefrom. The pump 30 of certain embodiments maintains a pressure at the anterior chamber 20 that is lower than that at Schlemm's canal 22 or downstream. In the first step, the inlet portion 33 is filled with aqueous fluid 24. In a second step, the pressure of the middle portion 36 is lowered to be below that of the inlet portion 33 so as to open the first check valve 35A, and aqueous fluid 24 flows into the middle portion 36 until the pressure equalizes between the inlet portion 33 and the middle portion 36. In a third step, the tube 37 of the middle portion 36 is compressed to push aqueous fluid 24 into the outlet portion 34. The lowering of the pressure of the middle portion 36 can be achieved by any conventional methods, for example, pulling the tube wall radially outwardly using the energy converted from piezoelectric or thermoelectric sources. Another method to lower the pressure within the middle portion 36 is by connecting the middle portion 36 to a suction pump located at a distance away from the pump body.

To maintain a constant pressure, the pumping volume (ΔV) for each stroke of certain embodiments of an implantable pump is dependent on the stroke frequency. For example, an ocular-pulse pump operating at approximately 72 cycle/minute (heart rate) pumps at a rate that equals the aqueous production rate for the eye (typically 2.4 microliters/minute); therefore,
ΔV=(2.4 microliters/minute)/(72 cycles/minute)=0.03 microliters/cycle

In another embodiment, a blink pressure-pulse driven pump operating at approximately 1 cycle/20 seconds pumps aqueous with a stroke volume of:
ΔV=(2.4 microliters/minute)/(3 cycles/minute)=0.8 microliters/cycle

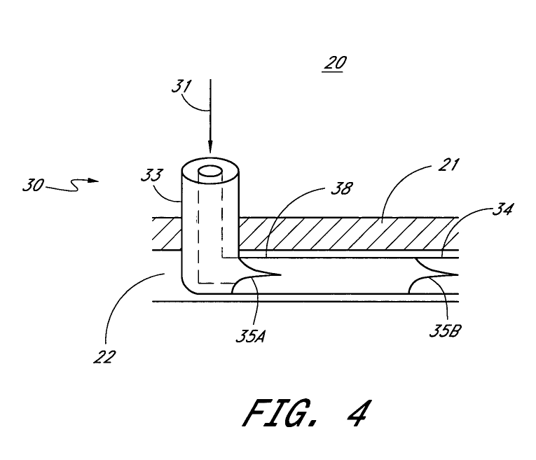

FIG. 4 schematically illustrates an embodiment of a pressure-pulse driven pump 30 implanted inside Schlemm's canal 22 of the eye 10. In one embodiment, pressure pulsations from the anterior chamber 20 press against the trabecular meshwork 21, which in turn presses against the flexible wall 38 of the middle portion 36 of the implanted pump 30. In another embodiment, pressure pulsations are converted into electricity via the piezoelectric mechanism and the electricity is used to drive a mechanical compressing unit for pressing against the flexible tube wall 38 of the middle portion 36. In certain embodiments, the outlet 34 is located inside Schlemm's canal 22 and aqueous fluid 24 exits the eye 10 through the collector channels and episcleral veins. Other variations include placing, or extending, the outlet 34 to the collector channels, aqueous veins 23, episcleral veins, and sub-conjunctival space. The aqueous fluid 24 (as shown by arrow 31) enters the inlet 33 of the pump 30 which is located in the anterior chamber 20 of the eye 10.

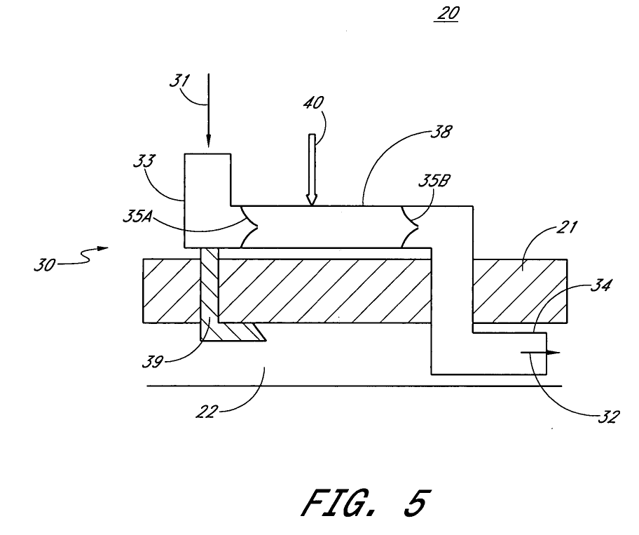

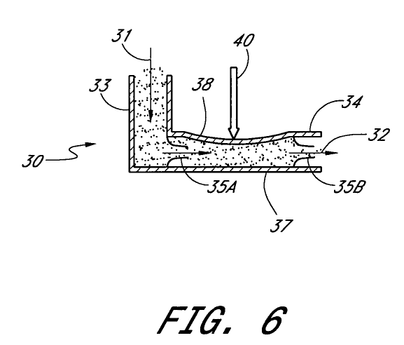

FIG. 5 schematically illustrates an embodiment of a pressure-pulse driven pump 30 located at an anterior angle 25 implant location. In this embodiment, the pump 30 is anchored into the trabecular meshwork 21 and Schlemm's canal 22 via an anchor 39 and the outlet 34. This configuration holds the pump 30 securely against the trabecular meshwork 21 in the anterior angle 25 of the eye 10. Alternatively, the anchor 39 of other embodiments is in other surrounding tissues and the pump 30 is placed in other parts of the eye 10, so long as it is exposed to or coupled to the driving pressure pulse 40. The inlet portion 33 is in the anterior chamber 20 and the outlet portion 34 is located in Schlemm's canal 22 or any of the various downstream structures. In addition, the outlet portion 34 of other embodiments is located in a vein within the iris 13 or eye wall. FIG. 6 schematically illustrates an overpressure prevention mechanism of certain embodiments of a dual-check valve pump 30. If the intraocular pressure exceeds the opening pressures of the valves 35A, 35B, then the pump 30 will allow free flow to regulate the intraocular pressure down into the desirable range. In this way, in certain embodiments, the pump 30 is designed to limit the maximum possible pressure that the eye 10 can achieve. This behavior may be particularly important during resting periods or periods of reduced cycle frequency during which the pump 30 may not be pumping at an adequate rate to keep up with the aqueous production.

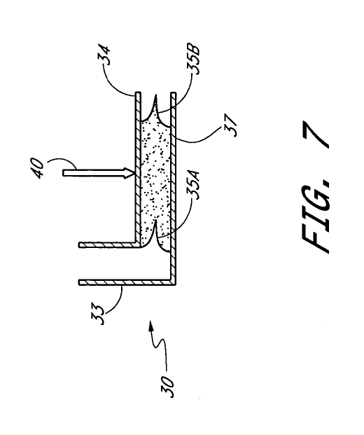

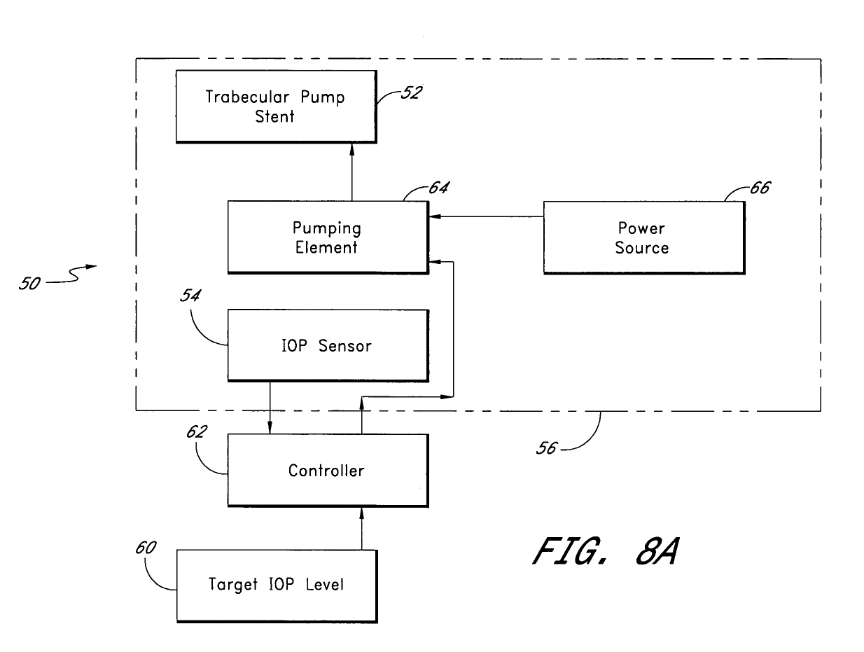

Complimentary to the over-pressure protection function, certain embodiments of the pump 30 also have a built-in under-pressure protection function as schematically illustrated in FIG. 7. It is desirable to not allow the intraocular pressure to drop below a low threshold, for example, 6 mmHg. Any intraocular pressure below the low threshold is considered hypotonous pressure and is dangerous to the eye 10 since it can cause choroidal hemorrhage, choroidal detachment, etc. The pump 30 is self-limiting, since the valves 35A, 35B will not open unless the pressure difference across the valve is greater than the opening pressure of the valve. If the maximum intraocular pressure is lower than the threshold pressure, then the valve will not open and aqueous fluid 24 will not leave the eye 10 through the outlet portion 34. The pump 30 will not function until the intraocular pressure rises above the threshold pressure of the valve through the inflow/production of aqueous fluid 24 from the ciliary body 16.

IOP Sensor and Transmitter

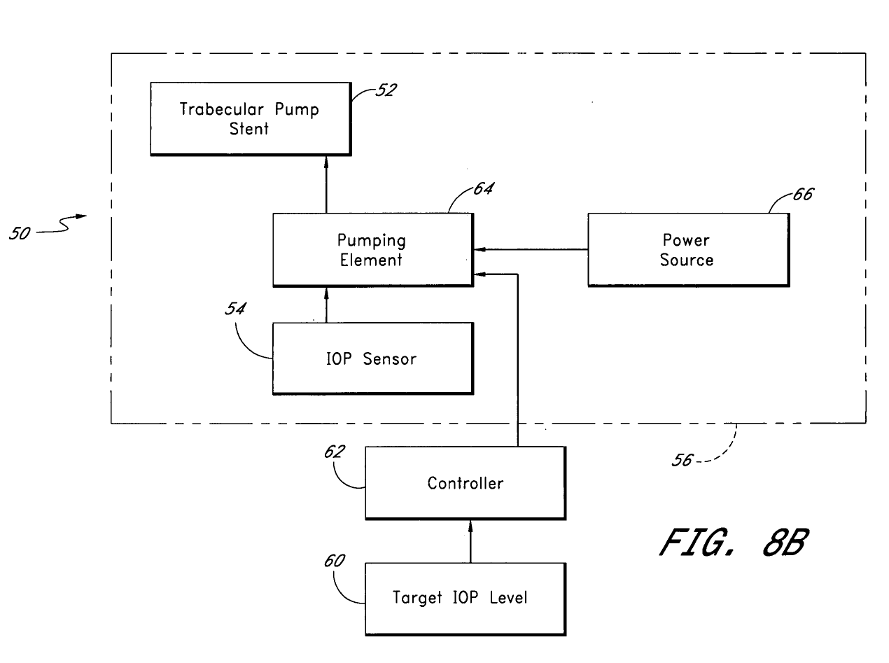

FIGS. 8A-8C are schematic block diagrams illustrating various embodiments of a trabecular pump stent system 50 for controlling the intraocular pressure of an eye 10. The trabecular pump stent system 50 comprises a trabecular pump stent 52, a pumping element 64 coupled to the trabecular pump stent 52, and a power source 66 coupled to the pumping element 64. The trabecular pump stent system 50 further comprises an intraocular pressure (IOP) sensor 54 and a controller 62. In certain embodiments, the block elements within the dashed line 56 of FIGS. 8A-8C are implanted within the eye 10.

In certain embodiments, the IOP sensor 54 is mounted on the trabecular pump stent 52, while in other embodiments, the IOP sensor 54 is spaced from the trabecular pump stent 52. The IOP sensor 54 is configured to measure an intraocular pressure and to generate a signal indicative of the measured pressure. This signal is then transferred to one or more other portions of the trabecular pump stent system 50 (e.g., the controller 62 or the pumping element 64). In certain embodiments, the IOP sensor 54 is configured to continuously generate and transmit the signal indicative of the measured intraocular pressure.

An exemplary IOP sensor 54 compatible with embodiments described herein is described in U.S. Pat. No. 6,579,235 to Abita et al., the entirety of which is hereby incorporated herein by reference. The IOP sensor 54 of certain embodiments comprises energy means for providing power to the IOP sensor 54. Examples of energy means compatible with embodiments described herein include, but are not limited to, a battery, piezoelectricity, and photovoltaic energy from microphotodiode cells. The IOP sensor 54 of certain embodiments further comprises sensing means for sensing the intraocular pressure and generating a signal indicative thereof. Examples of sensing means compatible with embodiments described herein include, but are not limited to, a combination of electronic circuitry and a mechanical sensing element such as a diaphragm displacement sensing unit, a capacitive sensor component, or a miniaturized pressure sensor as used for automobile air bags and in-situ tire gauges. The IOP sensor 54 of certain embodiments further comprises transmitting means for transmitting the signal to other portions of the trabecular pump stent system 50 (e.g., the controller 62 or the pumping element 64). In certain embodiments, the transmitting means comprises a radio frequency transmitter. In certain other embodiments, the transmitting means comprises a flashing LED (light emitting diode) which transmits a light signal indicative of the sensed IOP. The IOP sensor 54 of certain embodiments is configured to transmit the signal in response to an activation signal received from the controller 62.

For continuous monitoring of IOP, an IOP sensor 54 of certain embodiments comprises a capacitative-inductive circuit formed from a spiral inductor-diaphragm-based capacitor. An exemplary spiral inductor-diaphragm-based capacitor is disclosed by Cheryl Guttman in “Continuous IOP Monitoring Possible With Microsensor,” Ophthalmology Times, Oct. 15, 2003, which is incorporated in its entirety by reference herein. Upon sensing a change in the IOP level, the pressure-induced displacement of the diaphragm changes the frequency of the circuit. The IOP monitoring is performed telemetrically and taking a measurement does not require additional contact with the eye 10. In certain embodiments, the IOP sensor 54 utilizes an external pickup coil, which can be placed in an unobtrusive device, such as spectacles, to take a measurement form the circuit. The IOP sensor 54 of certain embodiments can vary from 1.3 mm to 6 mm in diameter, with resolutions of 1.2 to 1.4 mm Hg.

In certain embodiments, to achieve a target IOP level 60 prescribed for a patient, the trabecular pump stent 52 pumps aqueous fluid 24 out of the anterior chamber 20 toward Schlemm's canal 22 or downstream therefrom until the target IOP level 60 is reached. In certain embodiments, information regarding the target IOP level 60 is inputted to a controller 62. In certain embodiments, the controller 62 is remote from the other portions of the trabecular pump stent system 50 and comprises a microprocessor.

In the embodiment schematically illustrated in FIG. 8A, the controller 62 is outside the eye 10 and is configured to transmit wirelessly to an implanted pumping element 64 that is a part of the pump stent system 50. The pumping element 64 is coupled to an implanted power source 66, which provides power to the pumping element 64. Exemplary pumping elements 64 are described more fully below. Exemplary power sources 66 compatible with embodiments described herein include, but are not limited to, piezoelectric, photovoltaic, hydroelastic, and electrokinetic mechanisms, as described more fully below.

The controller 62 of certain embodiments is also configured to wirelessly receive signals from the IOP sensor 54 within the eye 10. The IOP sensor 54 is configured to generate a signal indicative of a sensed IOP and to transmit the signal to the controller 62. In certain such embodiments, the controller 62 is configured to compare the sensed IOP to the target IOP level 60 and to selectively activate the pumping element 64 in response to the signal. In this way, the trabecular pump stent 52 will function only when the sensed IOP is higher than the target IOP level 60.

For example, in certain embodiments, an external controller 62 is provided to take the IOP measurements from the IOP sensor 54 and is operatively coupled to an antenna in the IOP sensor 54. The controller 62 generates an activation signal for energizing the impedance element of the IOP sensor 54 to measure a signal representative of the intraocular pressure. This activation signal is preferably an electromagnetic signal that varies over a predetermined radiofrequency range (e.g., between approximately 100 MHz and approximately 200 MHz). The activation signal of certain embodiments energizes the circuit of the IOP sensor 54 and causes the circuit to resonate. The controller 62 of certain embodiments also includes a circuit to detect the resonant frequency of the IOP sensor 54 which is correlated to the intraocular pressure. This activation signal of certain embodiments is transmitted from the controller 62 multiple times over a predetermined time interval during which the IOP sensor 54 is in the anterior chamber 20.

In the embodiment schematically illustrated in FIG. 8B, the IOP sensor 54 is electrically coupled to the pumping element 64. The controller 62 of certain such embodiments is configured to transmit wirelessly to the pumping element 64. In certain such embodiments, the pumping element 64 receives the signal indicative of the sensed IOP from the IOP sensor 54 and a signal indicative of the target IOP level 60 from the controller 62. In one such embodiment in which the IOP sensor 54 transmits a light signal to the pumping element 64, the pumping element 64 comprises a transducer that converts the light signal into an electrical signal. The pumping element 64 of certain embodiments is activated when the sensed IOP is higher than the target IOP level 60. The pumping element 64 of certain such embodiments selectively activates to keep the sensed IOP substantially equal to the target IOP level 60.

Similarly, in the embodiment schematically illustrated in FIG. 8C, the IOP sensor 54 is electrically coupled to the power source 66. The controller 62 of certain such embodiments is configured to transmit wirelessly to the power source 66. In certain such embodiments, the power source 66 receives the signal indicative of the sensed IOP from the IOP sensor 54 and a signal indicative of the target IOP level 60 from the controller 62. The power source 66 of certain such embodiments selectively supplies power to the pumping element 64 to keep the sensed IOP substantially equal to the target IOP level 60. Other configurations of communication between the controller 62, the IOP sensor 54, the power source 66, and the pumping element 64 are compatible with embodiments described herein.

Certain embodiments provide a method of reducing the IOP of an eye. The method comprises setting a target IOP level 60 and sensing the real-time IOP. The method further comprises comparing the real-time IOP to the target IOP level 60. The method further comprises starting pumping aqueous fluid 24 out of the anterior chamber 20 once the real-time IOP is higher than the target IOP level 60.

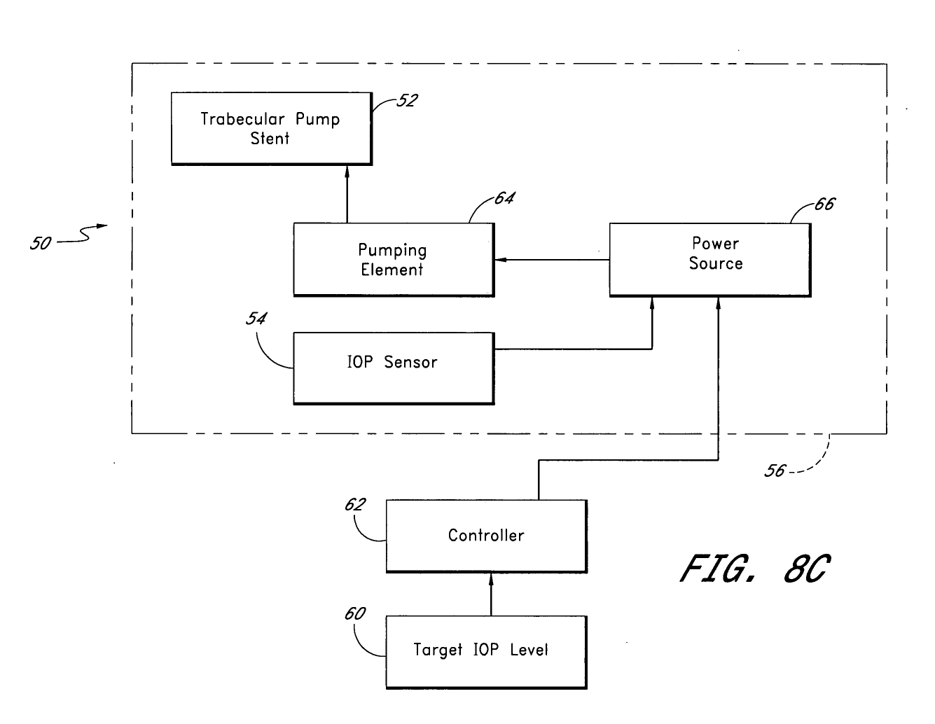

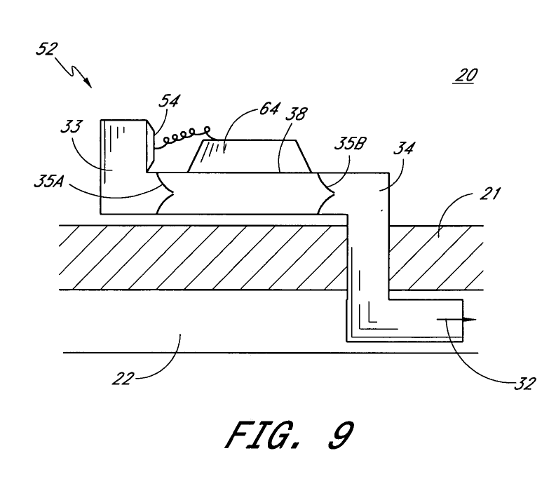

FIG. 9 schematically illustrates one embodiment of a pressure-pulse-driven trabecular pump stent 52 with an IOP sensor 54 for providing measured IOP data, and a pumping element 64. The pump stent 52 comprises an inlet portion 33, an outlet portion 34, and a middle portion 36 which is bordered by the first check valve 35A and the second check valve 35B. The IOP sensor 54 transmits a signal corresponding to the measured IOP to a pumping element 64. In certain embodiments, the pumping element 64 is intimately adhered to or wrapped around the wall 38 of the middle portion 36 and has the capability of providing suction to expand the tube wall 38 and providing pressure to compress the tube wall 38. The pumping element 64 can be powered by mechanical energy or electricity derived from various energy sources, including the conversion of mechanical to electrical energy. Exemplary energy sources for pumping elements 64 compatible with embodiments described herein include, but are not limited to, piezoelectric, photovoltaic, hydroelastic, and electrokinetic mechanisms, as described more fully below.

Piezoelectric Pumping Element

The piezoelectric effect is a phenomena resulting from a coupling between the electric and mechanical properties of a material. When mechanical stress is applied to a piezoelectric material, an electric potential is produced. Likewise, when an electric potential is applied to the material, a mechanical change occurs. Piezoelectric materials thus have numerous applications as electro-mechanical transducers—devices which can convert electrical signals into mechanical motion and vice-versa. Commercial applications of piezoelectric devices abound, for instance in speakers, spark generators inside electronic igniters, strain sensors, pressure gauges, and as precise time-keepers in electronic clocks.

In certain embodiments, a piezoelectric element is poled to generate the piezoelectric effect. Poling is a procedure in which the piezoelectric element is exposed to a poling voltage along the poling axis while at an elevated temperature, and then cooled while continuing to apply the poling voltage.

Piezoelectric substances produce an electric charge when a mechanical stress is applied to the substance, and produce a mechanical deformation when an electric field is applied to the substance. In one embodiment, the piezoelectric effect is exhibited by crystals which have no center of symmetry such as quartz and Rochelle salt. An important group of piezoelectric polycrystalline ceramics is ferroelectric materials with the perovskite crystal structure, such as barium titanate and lead zirconate titanate. Lead zirconate titanate ceramics and their modifications are solid solutions of lead titanate and lead zirconate. In certain embodiments, a 5-micron-thick ZnO piezoelectric layer is used to generate the piezoelectric effect. A few types of basic piezoelectric devices include crystals, tubes, unimorphs, bimorphs, and stacks.

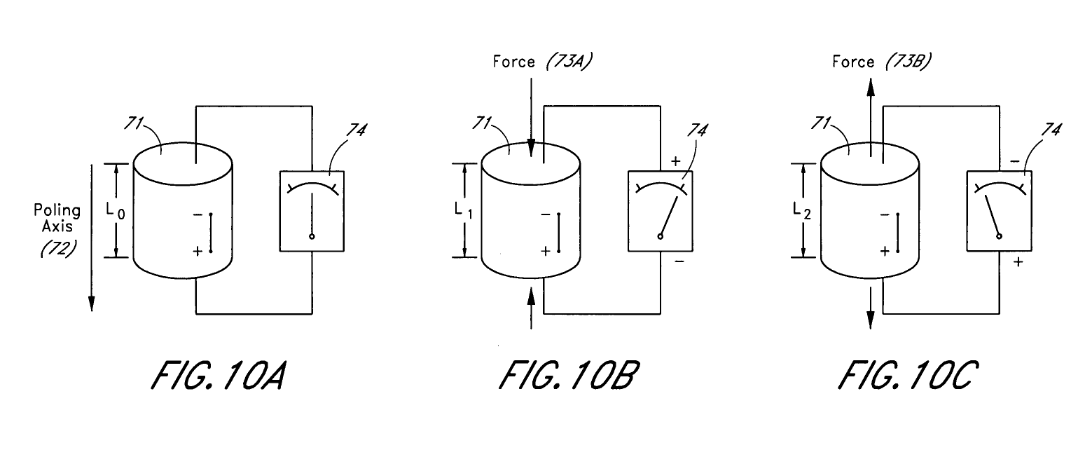

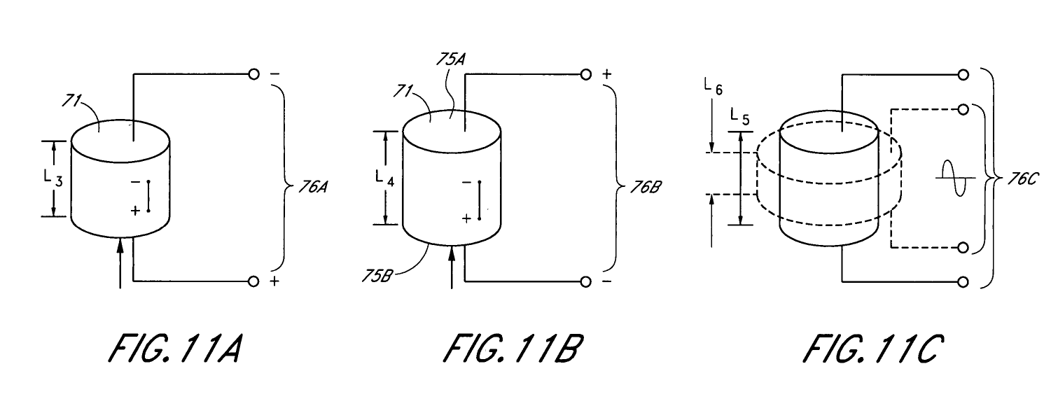

FIG. 10 schematically illustrates the piezoelectric effect utilized in certain embodiments to generate electrical charge from mechanical stress applied to a PZT (piezoelectric transducer) element. FIG. 11 schematically illustrates the piezoelectric effect utilized in certain embodiments to generate mechanical deformation from an electrical field applied to a PZT element.

As schematically illustrated in FIG. 10, a cylinder element 71 of PZT material in certain embodiments is placed at a suitable location to accept mechanical stress, for example under the eyelid with blink pressure pulses or in the anterior chamber with ocular pressure pulses. Similarly, in other embodiments, the cylinder 71 is placed at an appropriate location for accepting other mechanical stress, for example body motion, head motion or eye motion, to convert mechanical stress to piezoelectricity. Such embodiments use the PZT material in an analogous manner to the use by a battery-less watch that is powered by piezoelectric principles.

FIG. 10(a) shows the cylinder 71 under no-load conditions with a cylinder length of Lo along the direction of the poling axis 72. When an external force 73A produces mechanical stress or strain in the material, such that the cylinder 71 has a length which is different than Lo, the resulting change in the dipole moment causes a voltage 74 to appear between the electrodes on either side of the cylinder 71. If the cylinder 71 is compressed, the voltage will have the same polarity as the poling voltage, as schematically illustrated by FIG. 10(b). If the cylinder 71 is stretched by force 73B to have a length L₂ which is longer than L₁, the voltage 74 across the electrodes will have opposite polarity to the poling voltage, as schematically illustrated by FIG. 10(c). These examples illustrate piezoelectric generator action by which mechanical energy is converted into electrical energy. Examples of such piezoelectric generator action can be found in cigarette and gas lighters, gramophone pick-ups, accelerometers, hydrophones and microphones. In certain embodiments, the piezoelectric generator action is used for operating the pumping element 64 schematically illustrated by FIG. 9.

FIG. 11 schematically illustrates the piezoelectric conversion of electricity to mechanical deformation or movement of a PZT cylinder 71 having a cylinder length Lo and electrodes on a first surface 75A and on an opposing second surface 75B of the cylinder 71. The cylinder 71 will shorten to a cylinder length of L₃, which is less than L₀, if a voltage 76A of opposite polarity to the poling voltage is applied to the electrodes, as schematically illustrated by FIG. 11(a). If the applied voltage 76B has the same polarity as the poling voltage, then the cylinder 71 will lengthen to a cylinder length of L₄, which is longer than L₀, as schematically illustrated by FIG. 11(b). Finally, if an alternating voltage 76C is applied to the electrodes, the cylinder 71 will alternately extend to a length L₅ and shorten to a length L₆ with the same frequency as that of the applied voltage, as schematically illustrated by FIG. 11(c). These examples illustrate piezoelectric motor or actuator action by which electrical energy is converted into mechanical energy. Examples of such piezoelectric motor action can be found in piezoelectric transducers for ultrasonic cleaning equipment, ultrasonic atomizers, fuel injection systems and piezoelectric motors. In certain embodiments, the cylinder 71 is coupled to the driving pressure pulse 40 so that the cylinder 71 compresses the compressible tube 37 when the cylinder 71 lengthens. Certain embodiments provide a method of controlling IOP using piezoelectricity derived from blink pressure pulses, ocular pressure pulses, body motion, head motion and/or eye motion.

Certain embodiments utilize displacements far greater than those provided by simple transducers operating as shown in FIG. 11. By combining more than one piezoelectric element, certain embodiments further increase the amount of transduction. For instance, an elongating, bending or twisting device can be created by placing two layers of piezoelectric material on top of one another, and by controlling the polarization direction and the voltages such that when one layer contracts, the other will expand. Such a device is known as a bimorph. By stacking piezoelectric materials into layers, it becomes possible to combine their displacement to create a piezo stack. Such devices are capable of higher displacements and larger forces which are compatible with certain embodiments described herein.

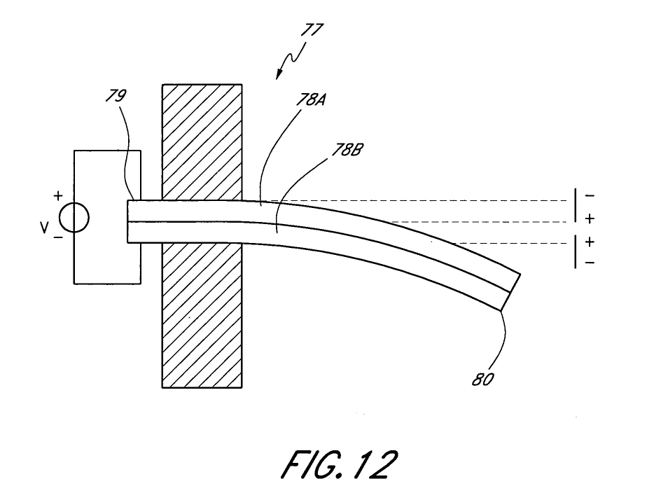

A much more compliant structure is the flexure element, which is the biomorph or bilaminar cantilever schematically illustrated in FIG. 12. FIG. 12 schematically illustrates the piezoelectric effect of a biomorph 77 comprising two thin PZT strips 78A, 78B bonded together. The two PZT strips 78A, 78B are mounted as a cantilever and are bonded together with their poling directions opposite to one another to form the biomorph 77. When voltage is applied to a first end 79 of the biomorph 77, the resulting piezoelectric polarity of the two PZT strips 78A, 78B causes the opposite second end 80 of the biomorph 77 to bend to a first direction. In certain embodiments, the biomorph 77 is coupled to the driving pressure pulse 40 so that the second end 80 of the biomorph 77 compresses the compressible tube 37 when the biomorph 77 bends.

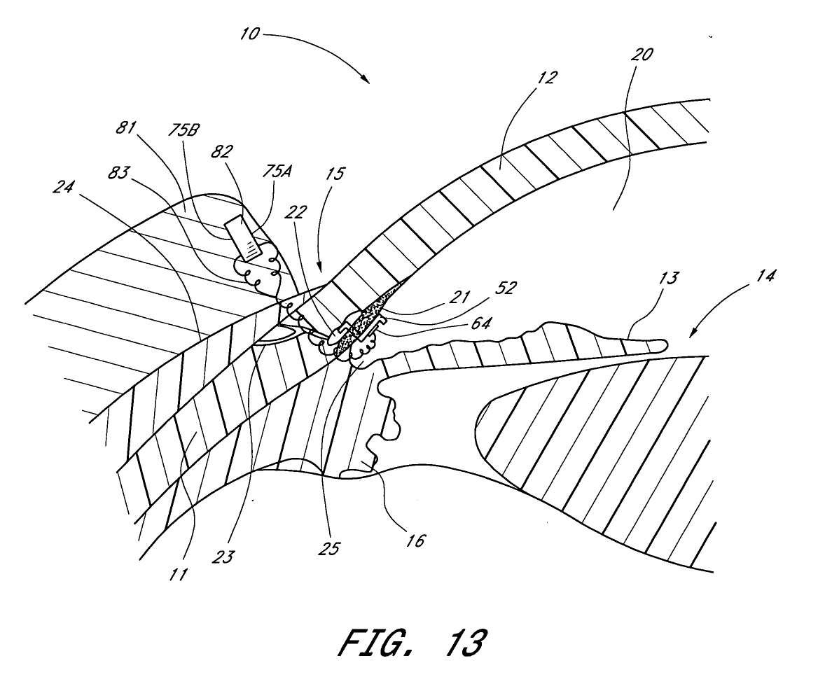

FIG. 13 schematically illustrates one embodiment of placing a PZT element 82 in an eye 10 for providing piezoelectricity to a trabecular pump stent 52. In certain embodiments, the PZT element 82 is placed at a lower eyelid 81, whereas the first surface 75A of the PZT element 82 faces the direction in which the upper eyelid approaches during the closing phase of the eye blink pulses. Conductors 83 are provided in certain embodiments to relay the piezoelectricity from the two opposite surfaces 75A, 75B of the PZT element 82 to the pumping element 64 of the trabecular pump stent 52. By setting the target IOP level 60 with a controller 62 wirelessly coupled to an IOP sensor 54, certain embodiments control the pumping element 64 so as to not activate the trabecular pump stent 52 upon reaching the target IOP level 60 (e.g., by turning off the power from the PZT element 82).

In one embodiment, the PZT element 82 comprises a cylinder with a biocompatible surface coating, such as silicone, Teflon, or phosphorylcholine, as disclosed in U.S. Pat. No. 5,599,587, which is incorporated in its entirety by reference herein. Phosphorylcholine is the name for the chemical head group found in the inner and outer layers of lipids forming the cell membranes. Phosphorylcholine contains both positive and negative charges and is electrically neutral overall (zwitterionic) over a wide pH range. As the predominant head group present in the lipids of the outer membrane layer, phosphorylcholine plays a key role in determining how the cell interacts with its surrounding environment. The zwitterionic nature of phosphorylcholine, combined with its ability to bind water tightly around itself, gives these lipids their remarkable biocompatibility.

Photovoltaic Cell

Certain embodiments provide a method for pumping fluid through a trabecular pump stent 52 in one direction. The method of certain embodiments comprises activating a pumping element 64 that is coupled to the pump stent 52, wherein the pumping element 64 is powered by electricity converted from power generated via a microphotodiode photovoltaic cell mechanism.

In certain embodiments, the power is generated from light entering through clear and translucent tissues of the eye 10. Microphotodiode photovoltaic cells have previously been incorporated on a subretinal microphotodiode-based silicone chip used in an artificial retina, as described in U.S. Pat. No. 5,024,223, which is incorporated herein in its entirety by reference. By way of example, the artificial retina prosthesis contains an array of about 5,000 microscopic photovoltaic cells on its surface. Each of the photovoltaic cells contains its own electrode and converts light energy into electrical impulses that are delivered to stimulate damaged, but still functional, overlying retinal cells. The principle of such microphotodiode photovoltaic cells is based on light of sufficient energy impinging onto a semiconductor material and promoting electrons into a high energy state and enabling transport of the electrons toward the n-type semiconductor material of a p-n type junction. The magnitude of the current generated depends on the intensity of the light impinging onto the cell.

Generally, photovoltaic cells are devices which convert light energy into electricity, either directly via the photovoltaic effect, or indirectly by first converting the light energy to heat or chemical energy. There is now a variety of methods for the practical production of silicon photovoltaic cells (amorphous, single crystal, polycrystalline), as well as photovoltaic cells made from other materials (copper indium diselenide, cadmium telluride, etc). Silicon photovoltaic cells are made using single crystal wafers, polycrystalline wafers or thin films.

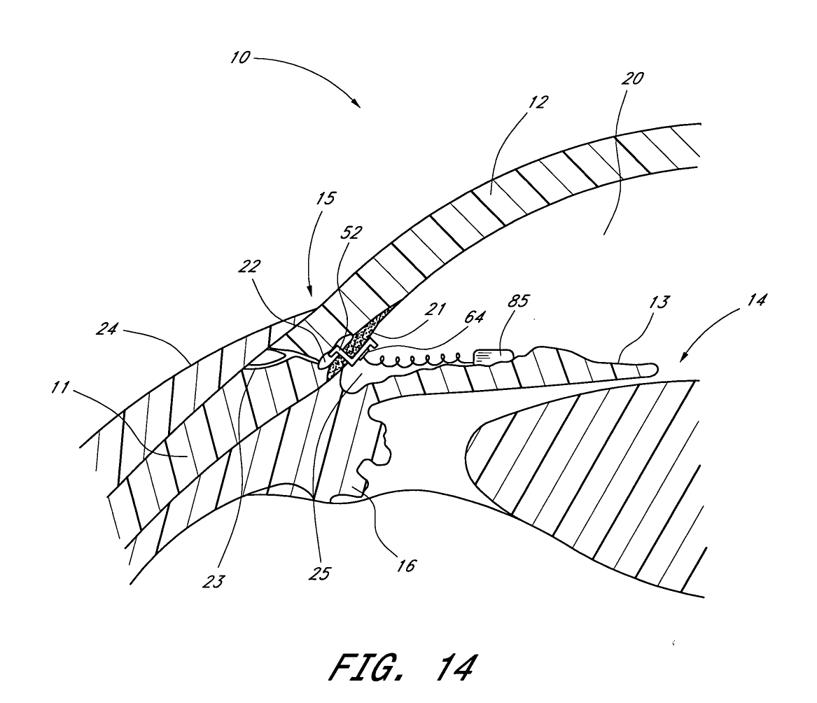

FIG. 14 schematically illustrates one embodiment of placing a photovoltaic cell array 85 on the iris 13 of an eye 10 for providing electricity generated from light energy to the pumping element 64 of a trabecular pump stent 52. In certain embodiments, the photovoltaic cell array 85 is placed in the anterior chamber 20 of the eye 10 by securing it onto the peripheral iris tissue out of the passageway of the light entering the pupil. In certain embodiments, the power output of a photovoltaic cell is increased quite effectively by using a tracking mechanism to keep the photovoltaic device directly facing the light. By setting the target IOP level 60 with a controller 62 connected to an IOP sensor 54, certain embodiments control the pumping element 64 so as to not activate the pump stent 52 upon reaching the target IOP level 60 (e.g., by turning off the power from the photovoltaic cell array 85).

Valveless IOP Pump

In certain embodiments, the IOP inside the anterior chamber 20 is controlled to be at a target level that is lower than the pressure in Schlemm's canal 22 or in the aqueous collector ducts. For certain such embodiments, a pump with IOP sensing and aqueous pumping functions is implanted in the eye. Certain embodiments use a hydroelastic pump formed from an asymmetric tube which is pinched to form asymmetric forces that pump fluid. Such hydroelastic pumps are described by U.S. Pat. Nos. 6,254,355 and 6,679,687, each of which is incorporated herein in its entirety by reference.

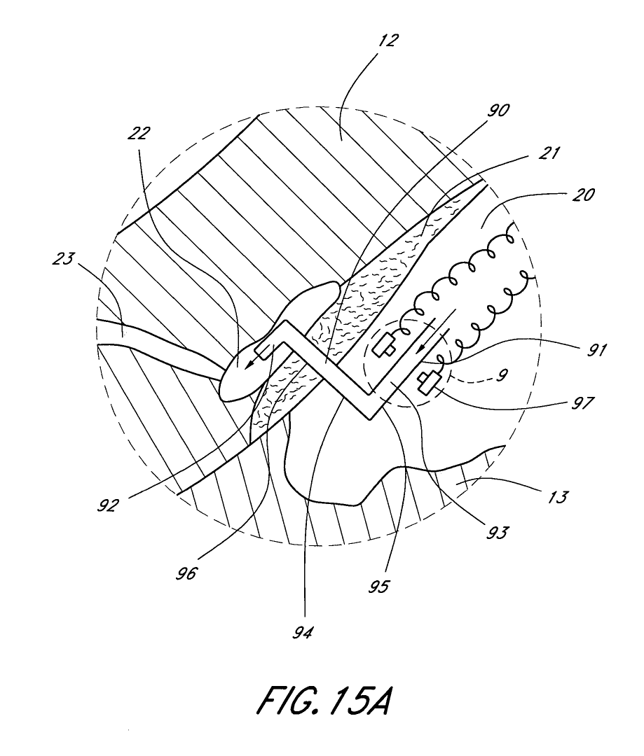

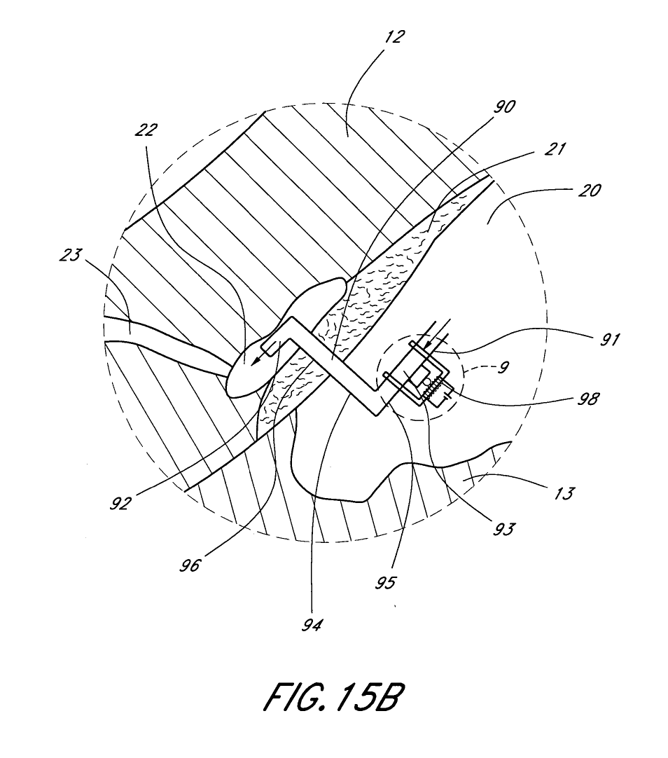

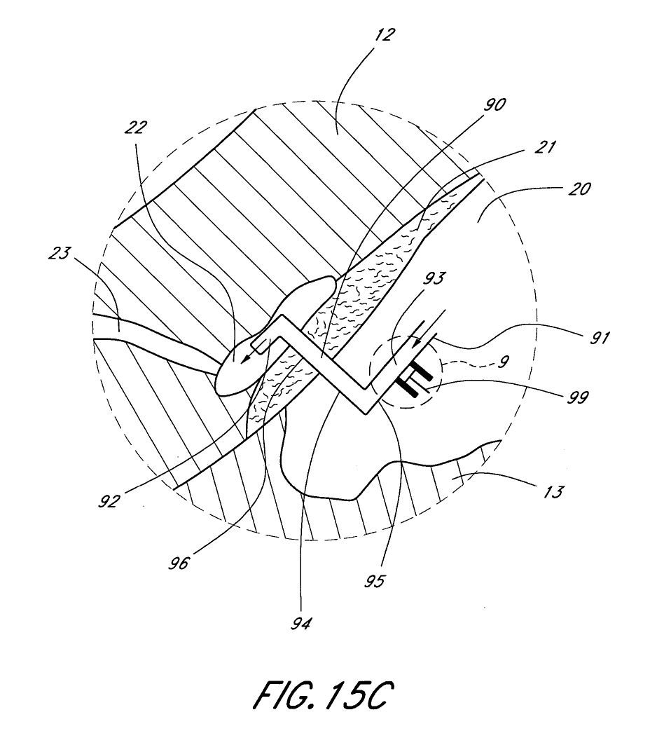

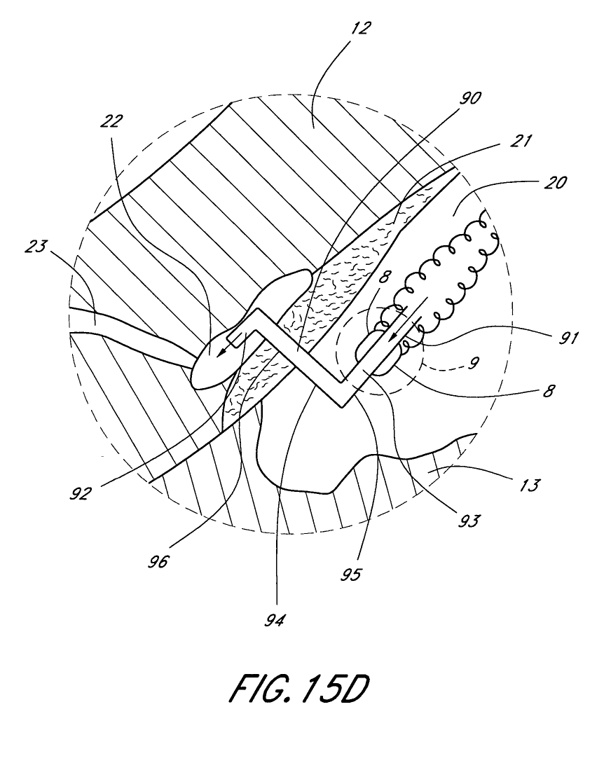

FIGS. 15A-15C schematically illustrate various exemplary embodiments of a pump 90 for transporting aqueous fluid from the anterior chamber 20 of an eye 10. In certain embodiments, the pump 90 comprises an inlet 91 configured to receive the aqueous fluid 24 from the anterior chamber 20, and an outlet 92 configured to output the aqueous fluid 24 to a location outside the anterior chamber 20 (e.g., Sclemm's canal 22). The pump 90 of certain embodiments also comprises a pump chamber 93 having a volume, the pump chamber 93 fluidly coupled to the inlet 91 and to the outlet 92. The volume of the pump chamber 93 is variable between at least first and second volumes.

As used herein, the term upump chamber refers to a space or cavity of the pump 90 which is at least partially enclosed. The pump chamber 93 of certain embodiments includes one or more openings through which aqueous fluid 24 can flow into or out of the pump chamber 93. The pump chamber 93 of various embodiments has a geometrical shape (e.g., disk or a tube), while in other embodiments, the pump chamber 93 has an irregular shape.

In certain embodiments, the volume of the pump chamber 93 is variable, or configured to be changed, so as to vary between at least a first volume value or a second volume value. In certain such embodiments, the volume of the pump chamber 93 is configured to be changed by pressure changes of the aqueous fluid 24 within the anterior chamber 20. Such exemplary embodiments are schematically illustrated by FIGS. 3A-3C, 4, 5, and 6. In other embodiments, the volume of the pump chamber 93 is configured to be selectively adjustable by a mechanism or an actuator, as described more fully below.

The pump chamber 93 of certain embodiments includes at least one movable portion (e.g., a movable wall) and upon moving this movable portion, the volume of the pump chamber 93 is changed. In certain embodiments, the movable portion of the pump chamber 93 comprises at least one elastic sidewall which at least partially encloses the pump chamber 93, and which is configured to expand or contract upon application of a corresponding force to the wall. In certain embodiments, the movable portions of the pump chamber 93 comprise a sidewall which at least partially encloses the pump chamber 93 and which translates relative to other portions of the pump chamber 93 upon application of a corresponding force to the wall to increase or decrease the volume of the pump chamber 93. Other movable portions of the pump chamber 93 which vary the volume of the pump chamber 93 upon movement are compatible with embodiments described herein.

In certain embodiments, the pump chamber 93 of the pump 90 comprises a tubular conduit 94 wherein at least a portion of the tubular conduit 94 is positionable to traverse the trabecular meshwork 21. FIGS. 3A-3C, 4, 5, and 6 schematically illustrate embodiments comprising a first check valve 35A at a first position along the tubular conduit 94 and a second check valve 35B at a second position along the tubular conduit 94. In such embodiments, the volume of the pump chamber 93 is fluidly coupled to the first check valve 35A and fluidly coupled to the second check valve 35B. The elastic sidewall of certain such embodiments is positioned between the first check valve 35A and the second check valve 35B.

In certain embodiments, the tubular conduit 94 has a first tubular portion 95 and a second tubular portion 96. The first tubular portion 95 is fluidly coupled to the inlet 91 and has a first fluidic characteristic. The second tubular portion 96 is fluidly coupled to the first tubular portion 95, is fluidly coupled to the outlet 92, and has a second fluidic characteristic. The second fluidic characteristic is different than the first fluidic characteristic.

In certain embodiments, the first fluidic characteristic is a first length and the second fluidic characteristic is a second length different from the first length. In certain embodiments, the first fluidic characteristic is a first elasticity and the second fluidic characteristic is a second elasticity different from the first elasticity. In certain embodiments, the first fluidic characteristic is a first diameter and the second fluidic characteristic is a second diameter different from the first diameter. In certain embodiments, the first fluidic characteristic is a first resistance to fluid flow and the second fluidic characteristic is a second resistance to fluid flow different from the first resistance. In other embodiments, the first fluidic characteristic results from a first combination of a first length, first elasticity, and/or first diameter which is different from the second fluidic characteristic resulting from a second combination of a second length, second elasticity, and/or second diameter.

The pump 90 of certain embodiments further comprises an actuator or pumping element 97 outside the tubular conduit 94, as schematically illustrated by FIGS. 15A-15C. The pumping element 97 of certain such embodiments is configured to change the volume of the pump chamber 93 and to generate a pressure difference between the first tubular portion 95 and the second tubular portion 96, thereby causing a hydroelastic pumping action based on the pressure difference. The pressure difference pumps the aqueous fluid 24 from the inlet 91, through the tubular conduit 94, and through the outlet 92.

In certain embodiments, at least one of the first tubular portion 95 and the second tubular portion 96 comprises an elastic sidewall coupled to the pumping element 97. In the embodiments schematically illustrated by FIG. 15A-15C, the pumping element 97 comprising a compression pinching segment coupled to the first tubular portion 95. The compression pinching segment (such as the piezoelectric pumping element 97 of FIG. 15A, the electromagnetic pumping element 98 of FIG. 15B, and the piston/cylinder pinching element 99 of FIG. 15C) is generally made of biocompatible material, is surface coated with biocompatible material, or is enclosed within a porous or meshed biocompatible enclosure 9 adapted for eye implantation. Furthermore, in certain embodiments, the compression pinching segment is sized, configured, and mounted at an appropriate location so that the compression pinching segment (along with its biocompatible enclosure 9, if so provided), neither obstructs the incoming light nor contact the iris 13.

In certain such embodiments, the pumping element 97 compresses a portion of the first tubular portion 95 to create a partial obstruction in the first tubular portion 95. In other embodiments, the pumping element 97 compresses a portion of the first tubular portion 95 to create a complete obstruction in the first tubular portion 95.

In other embodiments, the pump 90 comprises an adjustable diaphragm pump system, as schematically illustrated by FIG. 15D. In such embodiments, the variable volume of the pump chamber 93 is fluidly coupled to the first tubular portion 95 and fluidly coupled to the second tubular portion 96. In certain such embodiments, the pump chamber 93 comprises a diaphragm 8 coupled to the pumping element 97. The diaphragm 8 is responsive to the pumping element 97 by moving between at least two positions to adjust the volume of the pump chamber 93. In certain other embodiments, the pump chamber 93 comprises an elastic wall coupled to the pumping element 97. The elastic wall is responsive to the pumping element 97 by moving between at least two positions to adjust the volume of the pump chamber 93. The pumping element 97 of certain embodiments is powered by a battery, piezoelectricity, or one or more photovoltaic cells.

In certain embodiments, the pumping element 97 comprises at least one piezoelectric element, as described above. FIG. 15A schematically illustrates a pumping element 97 comprising a piezoelectric pincher element. The piezoelectric element compresses the first tubular portion 95 in response to an electrical signal.

In FIG. 15B, the pumping element 97 comprises an electromagnetic element 98 (e.g., an electromagnet) with a substantially U-shaped yoke and a pincher element. The electromagnet, when activated remotely from outside of the eye by an electromagnetic field, provides a magnetic force that pulls the pincher element on the first tubular portion 95 of the pump 90. U.S. Pat. No. 6,679,687, the entirety of which is incorporated herein by reference, discloses a magnet driving pump system that can be advantageous, for many reasons, as an implantable ocular pump.

FIG. 15C schematically illustrates a pump 90 having a pumping element 97 comprising a “T” shaped piston/cylinder pinching mechanism 99. The piston/cylinder pinching mechanism or element 99 may be powered by an implanted battery, piezoelectricity, photovoltaic cells, or other power sources. In each of the embodiments of FIGS. 15A-15C, the pumping element 97 applies force from outside of the pump 90 without a moving valve, propeller or blade hindering the fluid flow. Other embodiments utilize actuators or pumping elements 97 with other physical mechanisms to compress at least a portion of the first tubular portion 95. In other embodiments, the pumping element 97 is electrically coupled to an IOP sensor 54 and a controller 62 as described herein.

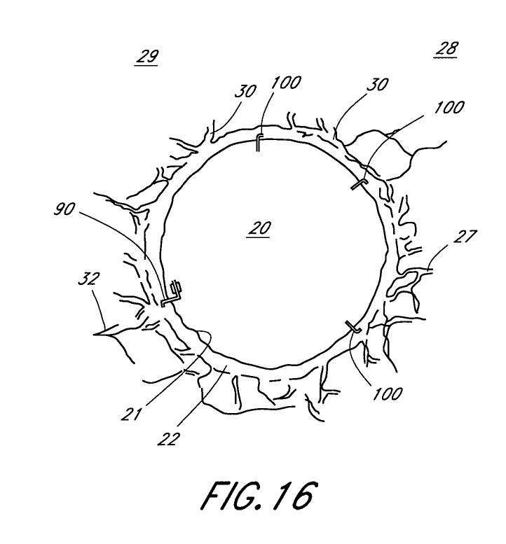

FIG. 16 schematically illustrates an apparatus 101for transporting aqueous fluid from the anterior chamber 20 of an eye 10. The apparatus 101 comprises a pump 90 and at least one implant 100. The implant 100 has an inlet 33 configured to receive aqueous fluid 24 from the anterior chamber 20 and an outlet 34 configured to output aqueous fluid 24.

In certain embodiments, the pump 90 and the implant 100 are separate components. In certain such embodiments, the pump 90 is configured to output the aqueous fluid 24 at a first location, and the implant 100 is configured to output aqueous fluid 24 to a second location different from the first location. In certain such embodiments, the pump 90 and the implant 100 are implanted within the eye at positions spaced around the circumference of the anterior chamber 20. In certain other embodiments, the pump 90 and the implant 100 are unitary, with the pump 90 in the implant 100 or on the implant 100.

Schlemm's canal 22 and collector channels 30 (collectively known as “aqueous cavities”) and arteries 32 in relation to the nasal side 29 and the temporal side 28 of the eye are schematically illustrated in FIG. 16. Aqueous fluid 24 enters Schlemm's canal 22 through the trabecular meshwork (not shown) and travels along Schlemm's canal 22 to exit through the collector channels 30. During the day, the pump 90 is activated by a power source (e.g., light energy or piezoelectricity) to maintain the target IOP level 60 which may be lower than the aqueous pressure in the aqueous cavities. During the evening, when the pump 90 does not function, aqueous fluid 24 is constantly produced by ciliary bodies 16. When the IOP in the anterior chamber 20 is higher than the aqueous pressure in the aqueous cavities, aqueous fluid 24 flows through the at least one implant 100 or through the pump 90 to maintain the essentially equivalent pressure.

Valveless Electrokinetic Pump

When an electrolyte solution passes over an insulating surface, the static charges present on the surface cause oppositely charged ions in solution to accumulate close to the surface. The bulk fluid motion combined with an excess of the oppositely charged ions results in a net flow of charge, which in turn induces a potential difference, called the “streaming potential.” The induced potential gradient forces oppositely charged ions, which are in excess close to the wall, to move upstream, thereby slowing down the bulk fluid or even causing a counter-flow close to the wall. If the solution is flowing through a small tube, the net effect is that the bulk fluid appears to have a higher viscosity due to the increase in flow resistance from conventional Poiseuille flow. This effect, known as the “electroviscous effect,” hinders the flow of liquid through nano- and micro- channels and tubes.

However, the same phenomenon may be exploited to enhance fluid flow in a weak or non-existent pressure gradient. In the presence of an externally applied electric field, the net movement of ions caused by the excess of ions close to the tube wall results in flow of the bulk fluid through the tube. The applied field thereby serves as the driving force for this pumping mechanism, commonly called an “electrokinetic pump.” Certain embodiments apply such pumping technology to the drainage of aqueous humor through a small tube from the anterior chamber of the human eye. Exemplary electrokinetic pumping compatible with embodiments described herein is disclosed by Denis J. Phares and Gregory T. Smedley in “A Study of Laminar Flow of Polar Liquids Through Circular Microtubes,” Physics of Fluids, Volume 16, No. 5, May 2004, pp. 1267-1272, which is incorporated herein in its entirety by reference.

U.S. patent application Publication No. 2003/0018295, the entire contents of which are incorporated herein by reference, discloses an electrokinetic medicament delivery device similar to a contact lens that is employed to therapeutically treat the conjunctiva for acute glaucoma. U.S. patent application Publication No. 2003/0010638, the entire contents of which are incorporated herein by reference, discloses a nanopump device for pumping small volumes of electrolyte solution under the control of a voltage source. The device includes a chamber and a nanopore membrane which partitions the chamber into an upstream region and a downstream region. When a voltage potential is applied across the membrane, electroosmotic flow through the membrane channels produces a precise-volume flow between the two chamber regions.

In certain embodiments, the governing equations for hydrodynamic and electrokinetic flows are used to investigate the feasibility of draining aqueous humor through a small tube from the anterior chamber of the human eye to Schlemm's canal or other outflow pathways. In certain embodiments, a trabecular stent has an 80 micron inner diameter lumen that spans the thickness of the trabecular meshwork (100 microns). Aqueous humor is a liquid very similar to plasma in that the only ions present in significant amounts are sodium (163 mM), chloride (132 mM), and bicarbonate (20.2 mM). Assuming the fluid to be saline (NaCl) with equal ion concentrations of 160 mM, a calculation of the expected flow rate can be made. An applied field of 10,000 volts/meter, which is equivalent to a one-volt potential difference across the stent (i.e. across the trabecular meshwork), would result in a net flow of 2.4 microliter/minute. This amount of flow is equal to the volumetric production rate of aqueous humor by the ciliary processes. Such a pump is therefore capable of sufficiently draining the anterior chamber even in the absence of a pressure gradient to drive the conventional outflow.

In such embodiments, an applied field of 10,000 volts/meter across the stent would induce a current of sodium and chloride ions equal to 6.4 microamps. For an applied potential drop of 1 volt across the stent, 6.4 microwatts of power must be supplied to the pump to sustain the potential gradient across the stent.

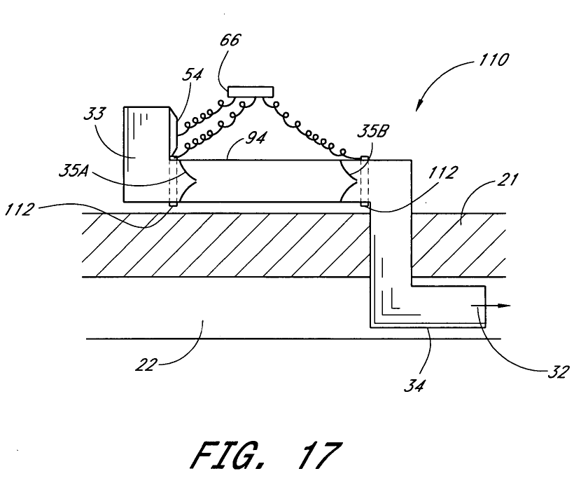

FIG. 17 schematically illustrates one embodiment of a membrane-less electrokinetic pump implant 110 for drainage of aqueous fluid 24. The pump 110 schematically illustrated by FIG. 17 comprises a tube 111 having an inlet 33 configured to receive the aqueous fluid 24 from the anterior chamber 20 and having an outlet 34 configured to output the aqueous fluid 24 to a location outside the anterior chamber 20. The tube 111 has a wall 113 with an inner surface 115A and an outer surface 115B. The inner surface 115A of the tube 111 defines a flow path between the inlet 33 and the outlet 34. The pump 110 further comprises at least one electrode 112 configured to generate an electric field along and generally parallel to at least a portion of the flow path. In certain embodiments, the electric field induces flow of aqueous fluid 24 from the inlet 33 to the outlet 34.

In certain embodiments, the tube 111 has an axis and has a generally circular cross-section perpendicular to the axis of the tube 111. In other embodiments, the cross-section of the tube 111 is non-circular (e.g., oval, rectangular, polygonal, asymmetric). The tube 111 cross-section of certain embodiments is different for different sections along the tube 111. The tube 111 of certain embodiments is curved or has bends such that sections of the tube 111 are non-parallel to one another.

In the embodiment schematically illustrated by FIG. 17, the pump 110 further comprises a first check valve 35A at a first position along the tube 111 and a second check valve 35B at a second position along the tube 111. Other embodiments comprise only a single check valve between the inlet 33 and the tube 111, while still other embodiments do not comprise a check valve at all.

The pump 110 schematically illustrated by FIG. 17 is coupled to an IOP sensor 54, which transmits a signal indicative of the sensed IOP to a power source 66. The power source 66 is operatively connected to the electrodes 112 for applying a selected voltage potential across the flow. The power sources 66 of certain embodiments is configured to selectively apply the voltage to the electrodes 112 in response to the signal from the IOP sensor 54. Power sources 66 compatible with embodiments described herein utilize various mechanisms to generate power, including, but not limited to, piezoelectricity, light energy via a microphotodiode cell mechanism, battery, isotope energy via isotope decay mechanism, or the like.

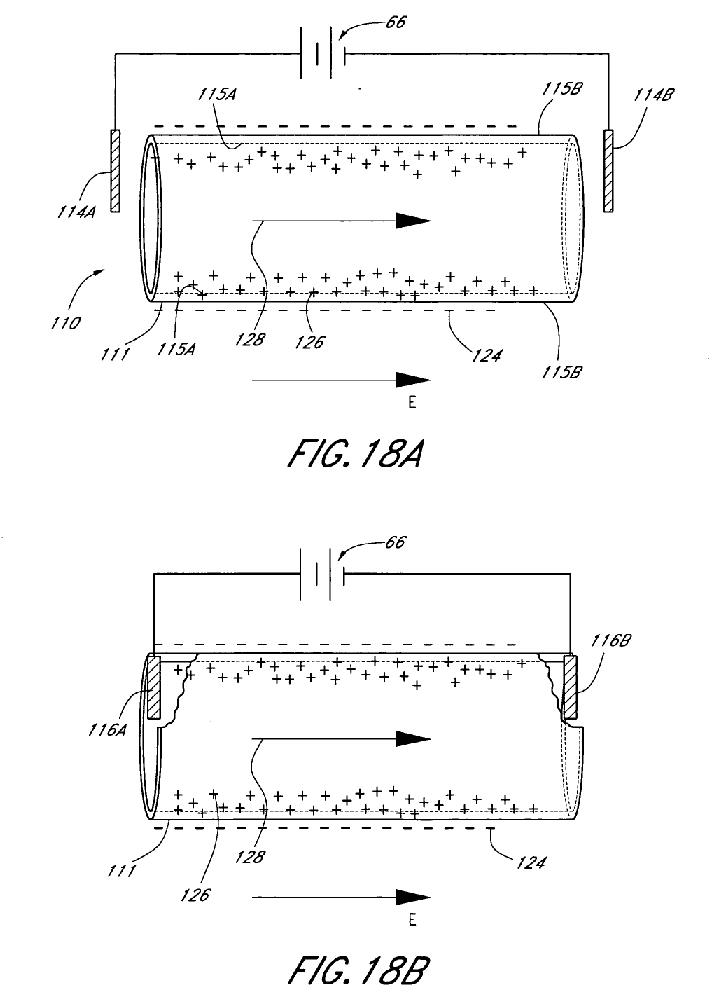

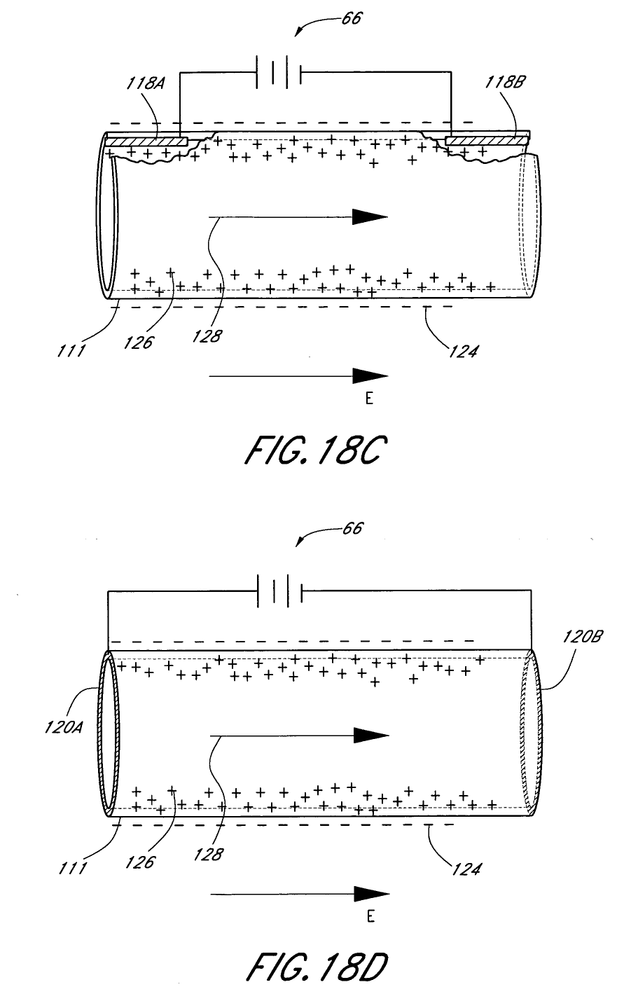

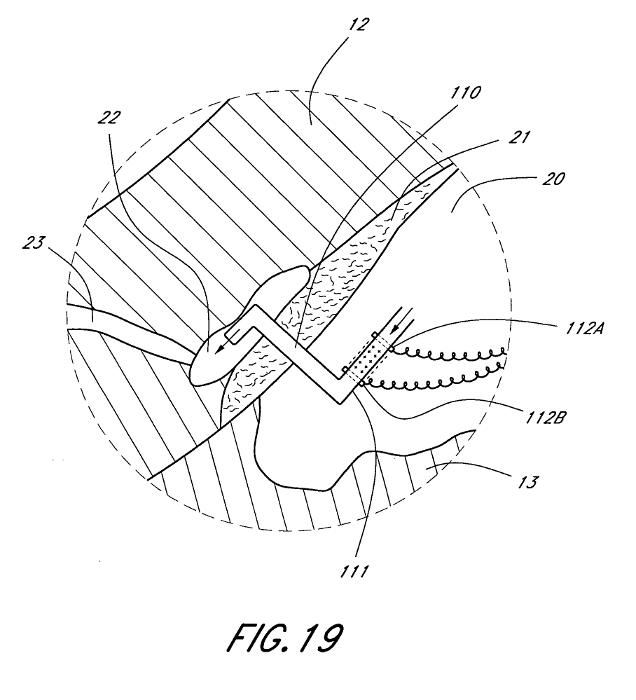

FIGS. 18A-18D schematically illustrate various embodiments of a tube 111 of a pump 110 utilizing electrokinetic pumping. In certain embodiments, at least a portion of the inner surface 115A of the tube 111 is electrically insulative. The first electrode 114A is at a first position along the tube 111 and the second electrode 114B is at a second position along the tube 111. The second position is spaced from the first position. In certain embodiments, the second position is at least 10 microns from the first position, while in other embodiments, the second position is at least 100 microns from the first position. In still other embodiments, the second position is spaced from the first position such that an electric field is applied along at least 10% of a flow path of aqueous fluid from the anterior chamber 20 to a location outside the anterior chamber 20. In certain embodiments, the electric field is applied along at least 30% of the flow path, along at least 50% of the flow path, along at least 80% of the flow path, or along approximately 100% of the flow path. In certain embodiments, the flow path is defined to be from a first portion of the implant which receives aqueous fluid 24 from the anterior chamber 20 to a second portion of the implant which outputs the aqueous fluid 24 to a position outside the anterior chamber 20.

In the embodiment schematically illustrated by FIG. 18A, the electrodes 114A, 114B are spaced from the tube 111 and extend generally perpendicularly to the tube 111. In the embodiment schematically illustrated by FIG. 18B, the electrodes 116A, 116B are in contact with the tube 111 and the first electrode 116A extends generally perpendicularly to a first portion of the tube 111 and the second electrode 116B extends generally perpendicularly to a second portion of the tube 111. In the embodiment schematically illustrated by FIG. 18C, the electrodes 118A, 118B are in contact with the tube 111. The first electrode 118A extends along at least a first portion of the tube 111 and the second electrode 118B extends along at least a second portion of the tube 111. In the embodiment schematically illustrated by FIG. 18D, the first electrode 120A has a generally annular shape configured to allow aqueous fluid 24 to flow therethrough and the second electrode 120B has a generally annular shape configured to allow aqueous fluid 24 to flow therethrough. In certain embodiments, the electrodes are positioned within the tube 111, while in other embodiments, the electrodes are positioned outside the tube 111. In still other embodiments, the electrodes are part of the wall 113 of the tube 111. Other configurations, shapes, and numbers of electrodes are also compatible with embodiments described herein.

As schematically illustrated in FIG. 18A, the pump 110 is activated by applying a voltage across the electrodes 114A, 114B which generates an electric field E in a direction along and generally parallel to at least a portion of the flow path. The outer surface 115B of the tube 111 maintains static charges 124 on the outer surface 115B while the excess ions 126 accumulate near the inner surface 115A of the tube 111 so as to cause fluid flow in one direction 128 in FIGS. 18A-18D.

FIG. 19 schematically illustrates a placement of a pump 110 utilizing electrokinetic pumping in an eye 10 for controlling the target IOP. In certain embodiments, the electrodes of the pump 110 each have a generally annular shape which surrounds the tube 111. When energized, the flow starts inside the tube 111 of the pump 110 without a moving valve, propeller or blade hindering the fluid flow.

In certain embodiments, the pump 110 is part of an apparatus comprising an implant 100. In certain embodiments, the pump 110 and the implant 100 are separate components. In certain such embodiments, the pump 110 is configured to output the aqueous fluid 24 at a first location, and the implant 100 is configured to output aqueous fluid 24 to a second location different from the first location. In certain such embodiments, the pump 110 and the implant 100 are implanted within the eye 10 at positions spaced around the circumference of the anterior chamber 20. In certain other embodiments, the pump 110 and the implant 100 are unitary, with the pump 110 in the implant 100 or on the implant 100.

From the foregoing description, it should be appreciated that a novel approach for sensing and controlling the IOP at a target level has been disclosed for regulating intraocular pressure. While the invention has been described with reference to specific embodiments, the description is merely illustrative and is not to be construed as limiting the invention. Various modifications and applications may occur to those who are skilled in the art without departing from the true spirit and scope of the invention, as described by the appended claims and their equivalents.

1. A pump for transporting aqueous fluid from the anterior chamber of an eye, the pump comprising:
an inlet configured to receive the aqueous fluid from the anterior chamber;
a chamber having a volume, the chamber fluidly coupled to the inlet, the volume of the chamber being variable between at least first and second volumes; and
an outlet fluidly coupled to the chamber, the outlet configured to output the aqueous fluid to a location outside the anterior chamber.

2. The pump of claim 1, wherein the location outside the anterior chamber is within Schlemm's canal.

3. The pump of claim 1, wherein the volume of the chamber is selectively adjustable.

4. The pump of claim 1, wherein the chamber comprises a tubular conduit and at least a portion of the tubular conduit is positionable to traverse the trabecular meshwork.

5. The pump of claim 4, wherein the pump further comprises a first check valve at a first position along the tubular conduit and a second check valve at a second position along the tubular conduit, the volume of the chamber fluidly coupled to the first check valve and fluidly coupled to the second check valve.

6. The pump of claim 5, wherein the tubular conduit comprises an elastic sidewall positioned between the first check valve and the second check valve.

7. The pump of claim 4, wherein the tubular conduit comprises a first tubular portion fluidly coupled to the inlet, the first tubular portion having a first fluidic characteristic, and a second tubular portion fluidly coupled to the first tubular portion and fluidly coupled to the outlet, the second tubular portion having a second fluidic characteristic different from the first fluidic characteristic.

8. The pump of claim 7, wherein the first fluidic characteristic is a first length and the second fluidic characteristic is a second length different from the first length.

9. The pump of claim 7, wherein the first fluidic characteristic is a first elasticity and the second fluidic characteristic is a second elasticity different from the first elasticity.

10. The pump of claim 7, wherein the first fluidic characteristic is a first diameter and the second fluidic characteristic is a second diameter different from the first diameter.

11. The pump of claim 7, wherein the first fluidic characteristic is a first resistance to fluid flow and the second fluidic characteristic is a second resistance to fluid flow, the second resistance different from the first resistance.

12. The pump of claim 7, wherein at least one of the first tubular portion and the second tubular portion comprises an elastic sidewall.

13. The pump of claim 7, wherein the pump further comprises an actuator configured to adjust the volume of the chamber and to generate a pressure difference between the first tubular portion and the second tubular portion, wherein the pressure difference pumps the aqueous fluid from the inlet, through the tubular conduit, and through the outlet.

14. The pump of claim 13, wherein the actuator is configured to selectively compress a portion of the first tubular portion.

15. The pump of claim 14, wherein the actuator is configured to selectively compress the portion of the first tubular portion to form a partial obstruction in the first tubular portion.

16. The pump of claim 13, wherein the chamber comprises a diaphragm coupled to the actuator, the diaphragm responsive to the actuator by moving between at least two positions to adjust the volume of the chamber.

17. The pump of claim 13, wherein the chamber comprises an elastic wall coupled to the actuator, the elastic wall responsive to the actuator by moving between at least two positions to adjust the volume of the chamber.

18. The pump of claim 13, wherein the actuator comprises a piezoelectric element.

19. The pump of claim 13, wherein the actuator comprises an electromagnetic element.

20. The pump of claim 13, wherein the actuator comprises a pincher.

21. The pump of claim 13, wherein the actuator comprises a piston.

22. An apparatus for transporting aqueous fluid from the anterior chamber of an eye, the apparatus comprising:
a pump comprising:
an inlet configured to receive the aqueous fluid from the anterior chamber;
a chamber having a volume, the chamber fluidly coupled to the inlet, the volume of the chamber being variable between at least first and second volumes, the chamber comprises a tubular conduit and at least a portion of the tubular conduit is positionable to traverse the trabecular meshwork, the tubular conduit comprises a first tubular portion fluidly coupled to the inlet, the first tubular portion having a first fluidic characteristic, and a second tubular portion fluidly coupled to the first tubular portion and fluidly coupled to the outlet, the second tubular portion having a second fluidic characteristic different from the first fluidic characteristic;
an outlet fluidly coupled to the chamber, the outlet configured to output the aqueous fluid to a location outside the anterior chamber; and
an actuator configured to adjust the volume of the chamber and to generate a pressure difference between the first tubular portion and the second tubular portion, wherein the pressure difference pumps the aqueous fluid from the inlet, through the tubular conduit, and through the outlet;
a controller electrically coupled to the actuator; and
at least one intraocular pressure sensor electrically coupled to the controller, the intraocular pressure sensor configured to generate a signal indicative of a sensed intraocular pressure and to transmit the signal to the controller, the controller configured to respond to the signal by selectively actuating the actuator.

23. The apparatus of claim 22, wherein the controller is wirelessly coupled to at least one of the actuator and the at least one intraocular pressure sensor.

24. The apparatus of claim 22, wherein the intraocular pressure sensor is spaced from the pump.

25. The apparatus of claim 22, wherein the intraocular pressure sensor is on the pump.

26. An apparatus for transporting aqueous fluid from the anterior chamber of an eye, the apparatus comprising:
a pump configured to output the aqueous fluid at a first location, the pump comprising:
an inlet configured to receive the aqueous fluid from the anterior chamber;
a chamber having a volume, the chamber fluidly coupled to the inlet, the volume of the chamber being variable between at least first and second volumes; and
an outlet fluidly coupled to the chamber, the outlet configured to output the aqueous fluid to a location outside the anterior chamber; and
at least one implant having an inlet configured to receive aqueous fluid from the anterior chamber and having an outlet configured to output aqueous fluid to a second location different from the first location.

27. A method of removing aqueous fluid from the anterior chamber of an eye, the method comprising:
receiving aqueous fluid from the anterior chamber in a first portion of a pump;
outputting the aqueous fluid from a second portion of the pump to a location outside the anterior chamber; and
varying a pump volume fluidly coupled to the first portion and fluidly coupled to the second portion, wherein adjusting the pump volume generates a pressure difference between the first portion and the second portion which pumps aqueous fluid from the first portion to the second portion.

28. The method of claim 27, further comprising implanting an implant having an inlet configured to receive aqueous fluid from the anterior chamber and having an outlet configured to output aqueous fluid.

29. The method of claim 28, wherein the pump is in or on the implant.

30. The method of claim 28, wherein the pump is separate from the implant.

31. A pump for transporting aqueous fluid from the anterior chamber of an eye, the pump comprising:
a tube having an inlet configured to receive the aqueous fluid from the anterior chamber and having an outlet configured to output the aqueous fluid to a location outside the anterior chamber, the tube having a wall with inner and outer surfaces, the inner surface defining a flow path between the inlet and the outlet; and
at least one electrode configured to generate an electric field along and generally parallel to at least a portion of the flow path.

32. The pump of claim 31, wherein the electric field induces flow of aqueous fluid from the inlet to the outlet.

33. The pump of claim 31, wherein at least a portion of the inner surface of the tube is electrically insulative.

34. The pump of claim 31, wherein the at least one electrode comprises a first electrode at a first position along the tube and a second electrode at a second position along the tube, the second position different than the first position.

35. The pump of claim 34, wherein the first electrode extends generally perpendicularly to a first portion of the tube and the second electrode extends generally perpendicularly to a second portion of the tube.

36. The pump of claim 34, wherein the first electrode has a generally annular shape configured to allow aqueous fluid to flow therethrough and the second electrode has a generally annular shape configured to allow aqueous fluid to flow therethrough.

37. The pump of claim 34, wherein the first electrode extends along at least a first portion of the tube and the second electrode extends along at least a second portion of the tube.

38. The pump of claim 31, wherein the pump further comprises a first check valve at a first position along the tube and a second check valve at a second position along the tube.

39. An apparatus for transporting aqueous fluid from the anterior chamber of an eye, the apparatus comprising:
a pump comprising:
a tube having an inlet configured to receive the aqueous fluid from the anterior chamber and having an outlet configured to output the aqueous fluid to a location outside the anterior chamber, the tube having a wall with inner and outer surfaces, the inner surface defining a flow path between the inlet and the outlet; and
at least one electrode configured to generate an electric field along and generally parallel to at least a portion of the flow path;
a controller electrically coupled to the at least one electrode; and
at least one intraocular pressure sensor electrically coupled to the controller, the intraocular pressure sensor configured to generate a signal indicative of the intraocular pressure and to transmit the signal to the controller, the controller configured to respond to the signal by selectively applying a voltage to the at least one electrode.

40. The apparatus of claim 39, wherein the intraocular pressure sensor is spaced from the pump.

41. The apparatus of claim 39, wherein the intraocular pressure sensor is on the pump.

42. An apparatus for transporting aqueous fluid from the anterior chamber of an eye, the apparatus comprising:
a pump configured to output aqueous fluid at a first location, the pump comprising:
a tube having an inlet configured to receive the aqueous fluid from the anterior chamber and having an outlet configured to output the aqueous fluid to a location outside the anterior chamber, the tube having a wall with inner and outer surfaces, the inner surface defining a flow path between the inlet and the outlet; and
at least one electrode configured to generate an electric field along and generally parallel to at least a portion of the flow path; and
at least one implant having an inlet configured to receive aqueous fluid from the anterior chamber and having an outlet configured to output aqueous fluid to a second location different from the first location.

43. A method of removing aqueous fluid from the anterior chamber of an eye, the method comprising:
receiving aqueous fluid from the anterior chamber in a first portion of a pump;
outputting the aqueous fluid from a second portion of the pump to a location outside the anterior chamber; and
transporting aqueous fluid from the first portion along a flow path to the second portion by generating an electric field along at least 10% of the flow path from the first portion to the second portion.

44. The method of claim 43, further comprising implanting an implant having an inlet configured to receive aqueous fluid from the anterior chamber and having an outlet configured to output aqueous fluid.

45. The method of claim 44, wherein the pump is in or on the implant.

46. The method of claim 44, wherein the pump is separate from the implant.
